(19) United States
(12) Patent Application Publication
(10) Pub. No.: US 2025/0312321 A1
(43) Pub. Date: Oct. 9, 2025

(54) TRANSIENT SIROLIMUS WITH FASL MICROGELS

(71) Applicant: iTOLERANCE, Inc., Miami, FL (US)

(72) Inventors: Sumitra M. GHATE, Indianapolis, IN (US); Camillo RICORDI, Miami, FL (US); James F. MARKMANN, Weston, MA (US)

(21) Appl. No.: 18/865,252

(22) PCT Filed: May 11, 2023
(86) PCT No.: PCT/US2023/066897

§ 371 Date: Nov. 12, 2024

Related U.S. Application Data
(60) Provisional application No. 63/364,607, filed on May 12, 2022.

(51) Int. Cl. A61K 31/436; A61K 31/415; A61K 31/505; A61K 31/52; A61K 35/39; A61K 38/00; A61K 47/10; A61K 47/69; A61P 37/06; C07K 14/705; C07K 16/28

(52) U.S. Cl. CPC A61K 31/436; A61K 31/415; A61K 31/505; A61K 31/52; A61K 35/39; A61K 47/10; A61K 47/6903; A61P 37/06; C07K 14/70575; C07K 16/2887; A61K 38/00

(57) ABSTRACT

Provided herein are methods of inducing immune tolerance to graft cells in a human patient in need thereof comprising administering to the patient (i) the graft cells. (ii) a chimeric FasL protein conjugated to a hydrogel, and (iii) transient sirolimus.

### CROSS-REFERENCE TO RELATED APPLICATIONS

This application claims the priority benefit of U.S. Provisional Application No. 63/364,607, filed on May 12, 2022, which is incorporated herein by reference in its entirety.

### REFERENCE TO SEQUENCE LISTING SUBMITTED ELECTRONICALLY

The content of the electronically submitted sequence listing (File name: 4999_003PC01_SequenceListing_ST26; Size: 8,373 bytes; and Date of Creation: May 5, 2023) filed with the application is incorporated herein by reference in its entirety.

### BACKGROUND

Transplant of allogeneic graft cells is limited by the need for chronic immunosuppression for graft survival against host immune attack. Achieving long-term survival of allogeneic grafts without chronic immunosuppression remains an elusive goal in clinical transplantation. Life-long immunosuppression carries significant risks of morbidity and mortality, including the risk of cancer, impairment in kidney function, and beta-cell toxicity and requires use of adjunctive therapies such as chronic prophylactic antiviral, antifungal, and antimicrobial agents to prevent infections. Removing chronic immunosuppression eliminates these risks and may also have tremendous beneficial impact in reducing emotional, psychosocial, quality of life, and heath care monitoring costs for patients undergoing graft cell transplantation. Thus, there is an unmet medical need for methods of transplanting graft cells without use of chronic immunosuppression.

### BRIEF SUMMARY OF THE INVENTION

Provided herein are methods transplanting allogenic graft cells that do not require chronic immunosuppression but achieve long-term survival of the grafts. The methods comprise administering the graft cells and Fas ligand (FasL) conjugated to a hydrogel (e.g., microgel) along with a transient regimen of sirolimus.

In some aspects, a method of inducing immune tolerance to graft cells in a human patient in need thereof comprises administering to the human patient (i) the graft cells, (ii) a chimeric FasL protein conjugated to a hydrogel, and (iii) sirolimus, wherein the sirolimus is administered for 36 weeks or less, optionally for 24 weeks or less.

In some aspects, the sirolimus is administered at an initial dose that maintains sirolimus blood trough level of about 4 ng/ml to about 16 ng/ml. In some aspects, the sirolimus is administered at an initial dose that maintains sirolimus blood trough level of about 9 ng/mL to about 16 ng/mL. In some aspects, the sirolimus is administered at an initial dose that maintains sirolimus blood trough level of about 4 ng/ml to about 15 ng/mL. In some aspects, the sirolimus is administered at an initial dose that maintains sirolimus blood trough level of about 9 ng/ml to about 15 ng/ml. In some aspects, the sirolimus is administered at an initial dose that maintains sirolimus blood trough level of about 9 ng/mL to about 13 ng/mL. In some aspects, the initial dose is administered for an initial period of about 12 weeks or less. In some aspects, the initial dose is administered for an initial period of about 1 week to about 12 weeks. In some aspects, the initial dose is administered for an initial period of about 6 weeks to about 12 weeks.

In some aspects, the sirolimus is administered in a tapering regimen for a tapering period. In some aspects, the tapering regimen is administered after the initial dose for the initial period. In some aspects, the tapering regimen occurs after the initial dose for the initial period and comprises a first tapering dose administered for a first tapering period and a second tapering dose administered for a second tapering period. In some aspects, the first tapering dose maintains sirolimus blood trough levels of about 4 ng/ml to about 11 ng/mL. In some aspects, the first tapering dose maintains sirolimus blood trough levels of about 4 ng/ml to about 11 ng/mL. In some aspects, the first tapering period is about 2 weeks or less. In some aspects, the first tapering period is about 1 week to about 2 weeks.

In some aspects, the second tapering dose maintains sirolimus blood trough levels of about 4 ng/mL to about 9 ng/mL. In some aspects, the second tapering dose maintains sirolimus blood trough levels of about 5 ng/ml to about 9 ng/ml. In some aspects, the second tapering period is about 2 weeks or less. In some aspects, the second tapering period is about 1 week to about 2 weeks.

In some aspects, the tapering regimen further comprises a third tapering dose administered for a third tapering period. In some aspects, the third tapering dose maintains sirolimus blood trough levels of about 3 ng/ml to about 7 ng/mL. In some aspects, the third tapering period is about 2 weeks or less. In some aspects, the third tapering period is about 1 week to about 2 weeks.

In some aspects, the tapering regimen further comprises a fourth tapering dose administered for a fourth tapering period. In some aspects, the fourth tapering dose maintains sirolimus blood trough levels of about 1 ng/mL to about 5 ng/ml. In some aspects, the fourth tapering period is about 2 weeks or less. In some aspects, the fourth tapering period is about 1 week to about 2 weeks.

In some aspects, the tapering regimen further comprises a fifth tapering dose administered for a fifth tapering period. In some aspects, the fifth tapering dose maintains sirolimus blood trough levels of about 0 ng/mL to about 3 ng/mL. In some aspects, the fifth tapering period is about 2 weeks or less. In some aspects, the fifth tapering period is about 1 week to about 2 weeks.

In some aspects, the tapering regimen comprises maintaining sirolimus blood trough levels of about 4 ng/mL to about 11 ng/ml for 2 weeks or less, maintaining sirolimus blood trough levels of about 4 ng/mL to about 9 ng/ml for 2 weeks or less, maintaining sirolimus blood trough levels of about 3 ng/ml to about 7 ng/mL for 2 weeks or less, maintaining sirolimus blood trough levels of about 1 ng/ml to about 5 ng/ml for 2 weeks or less, and/or maintaining sirolimus blood trough levels of about 0 ng/ml to about 3 ng/mL for 2 weeks or less.

In some aspects, the tapering regimen comprises maintaining sirolimus blood trough levels of about 7 ng/mL to about 11 ng/mL for 2 weeks or less, maintaining sirolimus blood trough levels of about 5 ng/mL to about 9 ng/mL for 2 weeks or less, maintaining sirolimus blood trough levels of about 3 ng/ml to about 7 ng/ml for 2 weeks or less, maintaining sirolimus blood trough levels of about 1 ng/mL to about 5 ng/ml for 2 weeks or less, and/or maintaining sirolimus blood trough levels of about 0 ng/ml to about 3 ng/ml for 2 weeks or less.

In some aspects, the tapering period is (i) about 12 weeks or less or (ii) about 6 weeks to about 12 weeks.

In some aspects, the methods provided herein further comprise administering to the subject mesenchymal stem cells, an anti-CD20 agent, and/or an anti-CD47 agent. In some aspects, the anti-CD20 agent is rituximab.

In some aspects, the graft cells are selected from peripheral blood mononuclear cells (PBMCs), bone marrow cells, hematopoietic stem cells, stem cells, stem-cell derived cells, mesenchymal stem cells, dendritic cells, dendritic cells pulsed with autoantigens, human beta cell products, pancreatic islet cells, alloislets, hepatocytes, and splenocytes. In some aspects, the graft cells are pancreatic islet cells or insulin producing stem cell-derived pancreatic islet cells. In some aspects, the graft cells are hepatocytes. In some aspects, the graft cells are stem cells or stem cell-derived cells. In some aspects, the graft cells are derived from a deceased donor. In some aspects, the graft cells are allogeneic.

In some aspects, the chimeric FasL protein comprises a FasL moiety and a streptavidin or avidin moiety. In some aspects, the chimeric FasL chimeric protein further comprises a linker between the FasL moiety and the streptavidin or avidin moiety. In some aspects, the chimeric FasL protein comprises a FasL moiety and a streptavidin moiety. In some aspects, the FasL moiety is a matrix metalloproteinase resistant FasL protein. In some aspects, the chimeric FasL protein comprises the amino acid sequence of SEQ ID NO:4. In some aspects, the chimeric FasL protein is conjugated to the hydrogel via biotin.

In some aspects, the hydrogel is a microgel. In some aspects, the microgel is about 125 microns to about 175 microns. In some aspects, the microgel is about 150 microns. In some aspects, the hydrogel is a polyethylene glycol (PEG) microgel. In some aspects, the hydrogel is engineered to display a biotin moiety.

In some aspects, the graft cells are not encapsulated by the hydrogel.

In some aspects, a 2:1 ratio of hydrogels: graft cells is administered.

In some aspects, the immune tolerance to graft cells is induced to treat type 1 diabetes. In some aspects, at least 5,000 islet equivalents per kilogram of the human patient are administered.

In some aspects, the immune tolerance to graft cells is induced to treat liver failure.

In some aspects, the graft cells and the chimeric FasL protein conjugated to a hydrogel are administered to the omentum.

In some aspects, the sirolimus is administered orally. In some aspects, the sirolimus is administered as an oral solution. In some aspects, the sirolimus is administered as an oral tablet. In some aspects, the sirolimus is administered once daily. In some aspects, the sirolimus administration begins on the same day or up to 5 days before the day that the graft cells and the chimeric FasL protein conjugated to a hydrogel are administered to the human subject.

In some aspects, the method further comprises administering a prophylactic antiviral and an antimicrobial while the sirolimus is administered. In some aspects, the prophylactic antiviral is famciclovir. In some aspects, the antimicrobial comprises sulfamethoxazole and/or trimethoprim.

Also provided herein are uses a chimeric FasL protein conjugated to a hydrogel for inducing immune tolerance in a human patient according to any method provided herein.

Also provided herein are chimeric FasL proteins conjugated to hydrogels for use in inducing immune tolerance according any method provided herein.

### BRIEF DESCRIPTION OF THE DRAWINGS/FIGURES

FIGS. 1A and 1B show local presentation of streptavidin (SA)-FasL presenting microgels for islet transplantation. FIG. 1A shows synthetic biotin-PEG microgels, generated by microfluidic cross-linking, capturing, and presenting SA-FasL. SA-FasL-presenting microgels and islets are immobilized on the surface of the omentum of non-human primates (NHPs) using autologous plasma and thrombin where they induce immune acceptance. FIG. 1B shows a schema describing donor and diabetic major histocompatibility complex (MHC) mismatched recipient NHPs and treatment protocol. SA-FasL-presenting microgels with transient sirolimus monotherapy (SA-FasL microgels, n=4); control microgels with transient sirolimus monotherapy (microgels, n=3). (See Example 1.)

FIGS. 2A-G show that SA-FasL-presenting microgels induce islet allograft acceptance. FIG. 2A shows Kaplan-Meir survival curves of islet allografts in SA-FasL microgel (n=4) and microgel (n=3); Mantel-Cox test, P=0.010. Mean graft survival times, SA-FasL microgel: >180 days, microgel: 27.7 days. FIG. 2B shows non-fasting blood glucose levels (mean, blue line; SEM, grey shadow, left axis) and daily total external insulin requirement (EIR) (mean, red bars; lower SEM, dark red bars, right axis) for SA-FasL microgel NHPs. Animals exhibited high blood glucose levels and external insulin demand after STZ induction but prior to transplant (defined as Post-STZ). After co-transplantation of deceased-donor islets and SA-FasL-microgels (Tx), NHPs rapidly became normoglycemic and had significantly reduced external insulin requirement. Animals reverted to hyperglycemic state after graft removal (blood glucose levels, purple lines, left axis; total external insulin requirement, tan bars, right axis). FIG. 2C shows blood glucose levels for SA-FasL-microgel NHPs after IV infusion of glucose before (Pre STZ) and after diabetes induction (Post-STZ), at 3 and 6 months post-transplantation, and post graft removal. FIG. 2D shows insulin (left axis) and C-peptide (right axis) levels in serum for SA-FasL microgel NHPs under fasting (F) and post-stimulation(S) before (naïve) and after diabetes induction, at 3 and 6 months post-transplantation, and post-graft removal (PGR). FIG. 2E shows non-fasting blood glucose levels (blue line; SEM grey shadow, left axis) and daily total external insulin requirement (mean, red bars; lower SEM, dark red bars, right axis) for microgel NHPs. After restoring normoglycemia following transplantation (Tx), NHPs became hyperglycemic and required higher external insulin around 1 month post-transplantation. FIG. 2F shows blood glucose after IV infusion in microgel NHPs during pre-diabetic state (Pre STZ), after diabetes induction (Post-STZ), and at 1 month post-transplantation. FIG. 2G shows insulin (left axis) and C-172 peptide (right axis) levels in serum for microgel NHPs under fasting (F) and post-stimulation(S) before and after diabetes induction and at 1 month post-transplantation. (See Example 1.)

FIGS. 3A-B show quantification of histological analyses of FoxP3⁺ cells over total cell counts and mean intensity in sections from microgel and SA-FasL microgel NHPs demonstrating increased frequency of T_{regs} in SA-FasL microgel NHPs (nested two-tailed t-test). Plots show data points for each NHP in a different color with a minimum of three representative images analyzed per NHP. (See Example 1.)

FIGS. 4A-J show that local delivery of SA-FasL-microgels does not alter peripheral blood lymphocyte populations. In these figures, the numbers of circulating (FIG. 4A) CD3⁺, (FIG. 4B) CD20⁺, (FIG. 4C) CD4⁺, (FIG. 4D) CD8⁺ cells, as well as naïve (Tn) (FIG. 4E) CD4⁺ and (FIG. 4F) CD8⁺, effector memory (EM) (FIG. 4G) CD4⁺ and (FIG. 4H) CD8⁺, and central memory (CM) (FIG. 4I) CD4+ and (FIG. 4J) CD8⁺ lymphocytes in SA-FasL microgel treated NHPs (dashed blue lines, mean, SEM, n=3-4) and microgel receiving recipients (red solid lines, mean, SEM, n=3). (See Example 1.)

FIGS. 5A-H show that SA-FasL-microgel recipients display no significant changes in pre- to post-transplant anti-donor MHC antibodies, CD4⁺ and CD8⁺ T cell proliferative responses to donor and third-party stimulators, and IFN-γ secreting cell numbers. FIGS. 5A and 5B show IgG responses (mean, individual points) to donor-specific MHC (FIG. 5A) class I and (FIG. 5B) class II epitopes for SA-FasL microgel (blue, n=3-4) and (FIG. 5D; red, n=3) NHPs, demonstrating no donor-specific activation in treated NHPs (class I: P=0.2904, class II: P=0.0754). No positive donor-specific antibodies to MHC I were detected in microgel NHPs (P=0.3292). Nonetheless these control NHPs generated antibodies against donor MHC II (P=0.0326). FIGS. 5C-F show Mixed lymphocyte reaction (mean, individual points) to CD4+ (FIG. 5C) donor and (FIG. 5D) third-party stimulators and CD8⁺ (FIG. 5E) donor and (FIG. 5F) third-party antigens for SA-FasL microgel (blue, n=3-4) and microgel (red, n=3) NHPs showing no differences in responses for the latter (CD4+donor: P=0.4200; CD4+third party: P=0.6949; CD8+donor: P=0.4145; CD8⁺ third party: P=0.7858). Microgel NHPs exhibited no responses against donor (P-0.6082) or third-party (P-0.0555) antigens in CD4+compartment, but CD8⁺ T cell responses were elevated against both donor (P=0.0057) and third party (P=0.0216) stimulators. FIGS. 5G and H show ELISpot IFN-γ counts (mean, individual points) for (FIG. 5G) donor and (FIG. 5H) third-party stimulation for the SA-FasL microgel (blue, n=3-4) and microgel (red, n=3) NHPs. No differences in frequency of IFN-γ-secreting cells in circulation between pre- and post-transplant time points for SA-FasL microgel (donor: P-0.2485; third-party: P=0.1445) or microgel NHPs (donor: P=0.0824; third-party: P=0.1413). Repeated measures ANOVA was used with pairwise comparisons to pre-transplant/day 0 values using Dunnett's test. (See Example 1.)

FIGS. 6A-E shows serum markers for liver and kidney function and sirolimus (“rapa”) serum levels in NHPs receiving SA-FasL or control microgels. FIGS. 6A-C show Longitudinal tracking of (FIG. 6A) blood urea nitrogen (BUN), (FIG. 6B) creatinine, and (FIG. 6C) Alanine Aminotransferase (ALT) levels (mean, SEM) in NHPs receiving SA-FasL microgels (blue, n=4), and control microgels (red, n=3). FIG. 6D shows longitudinal tracking of sirolimus (“rapa”) concentration (mean, SEM) in serum and demonstrates no differences in sirolimus levels between NHPs of SA-FasL microgels (dashed blue lines, n=4) and control microgels (red lines, n=3) NHPs (two-tailed Mann-Whitney test, p=0.63). FIG. 6E shows serum sirolimus levels for individual recipients. (See Example 1.)

FIG. 7 shows a schematic of the clinical study using iTOL-101 and temporary administration of sirolimus. (See Example 2.)

### DETAILED DESCRIPTION OF THE INVENTION

### Terminology

As used in the present disclosure and claims, the singular forms “a,” “an,” and “the” include plural forms unless the context clearly dictates otherwise.

Unless specifically stated or obvious from context, as used herein, the term “or” is understood to be inclusive. The term “and/or” as used in a phrase such as “A and/or B” herein is intended to include both “A and B,” “A or B,” “A,” and “B.” Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided. In this disclosure, “comprises,” “comprising,” “containing” and “having” and the like can mean “includes,” “including,” and the like; “consisting essentially of” or “consists essentially” are open-ended, allowing for the presence of more than that which is recited so long as basic or novel characteristics of that which is recited is not changed by the presence of more than that which is recited, but excludes prior art aspects.

As used herein, the terms “about” and “approximately,” when used to modify a numeric value or numeric range, indicate that deviations of up to 10% above and down to 10% below the value or range remain within the intended meaning of the recited value or range. It is understood that wherever aspects are described herein with the language “about” or “approximately” a numeric value or range, otherwise analogous aspects referring to the specific numeric value or range (without “about”) are also provided.

As used herein, the term “sirolimus” (also known as rapamycin) refers to a macrocylic lactone compound with the chemical name
3S,6R,7E,9R,10R,12R, 14S,15E, 17E, 19E,21S,23S,26R,27R,34aS)-9,10,12,13,14,21,22,23,24,25,26,27,32,33,34, 34a-hexadecahydro-9,27-dihydroxy-3-[(1R)-2-[(1S,3R,4R)-4-hydroxy-3-methoxycyclohexyl]-1-methylethyl]-10,21-dimethoxy-6,8,12, 14,20,26-hexamethyl-23,27-epoxy-3H-pyrido[2,1-c][1,4] oxaazacyclohentriacontine-1,5, 11,28,29 (4H,6H,31H)-pentone. This term includes, for example, the active compound sold under the brand name Rapamune.

As used herein “FasL” refers to “Fas ligand,” also known as tumor necrosis factor ligand superfamily member 6, Apoptosis antigen ligand (APTL), or CD95 ligand (CD95-L). The sequence of human FasL protein is

(SEQ ID NO: 7)
MQQPFNYPYPQIYWVDSSASSPWAPPGTVLPCPTSVPRRPGQRRP
PPPPPPPPLPPPPPPPPLPPLPLPPLKKRGNHSTGLCLLVMFFMV
LVALVGLGLGMFQLFHLQKELAELRESTSQMHTASSLEKQIGHPS
PPPEKKELRKVAHLTGKSNSRSMPLEWEDTYGIVLLSGVKYKKGG
LVINETGLYFVYSKVYFRGQSCNNLPLSHKVYMRNSKYPQDLVMM
EGKMMSYCTTGQMWARSSYLGAVFNLTSADHLYVNVSELSLVNFE
ESQTFFGLYKL.

Amino acids 1-80 of SEQ ID NO:7 are the cytoplasmic domain of human FasL. Amino acids 81-102 of SEQ ID NO:7 are the transmembrane domain of human FasL, and amino acids 103-281 of SEQ ID NO:7 are the extracellular domain of human FasL.

A “chimeric FasL protein” refers to a protein comprising a fusion of a FasL protein or a fragment thereof to a heterologous protein (e.g., streptavidin or avidin) or fragment thereof.

As used herein “biotin” (hexahydro-2-oxo-1H-thieno (3,4-d) imidazole-4-pentanoic acid) includes biotin-containing moieties that are able to bind to surfaces, such as cell surfaces, such as HS-biotin and EZ-Link™ Sulfo-HS-LC-Biotin (Pierce). Biotin and protein-reactive forms of biotin are available commercially.

As used herein, “hydrogel” refers to a water swollen polymer material. These include, e.g., water-swollen polymer networks, with dimensions much larger than a cell (such as >500 μm). A hydrogel typically is formed when an organic polymer (natural or synthetic) is cross-linked via covalent, ionic, or hydrogen bonds to create a three-dimensional open-lattice structure which entraps water molecules to form a gel. Examples of materials which can be used to form a hydrogel include macromer-based materials (including PEG macromers) assembled using different cross-linking methods (such as Michael-type addition, thiol-ene, click reactions, etc.), polysaccharides (such as alginate), polyphosphazines, and polyacrylates, or block copolymers such as Pluronics™ or Tetronics™, polyethylene oxide-polypropylene glycol block copolymers which can be cross-linked by temperature, free radical polymerization, click reactions or pH, respectively.

As used herein, “microgel” refers to hydrogels with smaller dimensions (such as on the order of 10s or 100s of μm).

The terms “polypeptide,” “peptide,” and “protein” are used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

A polypeptide, antibody, polynucleotide, vector, cell, or composition which is “isolated” is a polypeptide, antibody, polynucleotide, vector, cell, or composition which is in a form not found in nature. Isolated polypeptides, antibodies, polynucleotides, vectors, cell or compositions include those which have been purified to a degree that they are no longer in a form in which they are found in nature. In some aspects, an antibody, polynucleotide, vector, cell, or composition which is isolated is substantially pure. As used herein, “substantially pure” refers to material which is at least 50% pure (i.e., free from contaminants), at least 90% pure, at least 95% pure, at least 98% pure, or at least 99% pure.

“Percent identity” refers to the extent of identity between two sequences (e.g., amino acid sequences or nucleic acid sequences). Percent identity can be determined by aligning two sequences, introducing gaps to maximize identity between the sequences. Alignments can be generated using programs known in the art. For purposes herein, alignment of nucleotide sequences can be performed with the blastn program set at default parameters, and alignment of amino acid sequences can be performed with the blastp program set at default parameters (see National Center for Biotechnology Information (NCBI) on the worldwide web, ncbi.nlm.nih.gov).

As used herein, amino acids with hydrophobic side chains include alanine (A), isoleucine (I), leucine (L), methionine (M), valine (V), phenylalanine (F), tryptophan (W), and tyrosine (Y). Amino acids with aliphatic hydrophobic side chains include alanine (A), isoleucine (I), leucine (L), methionine (M), and valine (V). Amino acids with aromatic hydrophobic side chains include phenylalanine (F), tryptophan (W), and tyrosine (Y).

As used herein, amino acids with polar neutral side chains include asparagine (N), cysteine (C), glutamine (Q), serine(S), and threonine (T).

As used herein, amino acids with electrically charged side chains include aspartic acid (D), glutamic acid (E), arginine (R), histidine (H), and lysine (K). Amino acids with acidic electrically charged side chains include aspartic acid (D) and glutamic acid (E). Amino acids with basic electrically charged side chains include arginine (R), histidine (H), and lysine (K).

As used herein, the term “host cell” can be any type of cell, e.g., a primary cell, a cell in culture, or a cell from a cell line. In some aspects, the term “host cell” refers to a cell transfected with a nucleic acid molecule and the progeny or potential progeny of such a cell. Progeny of such a cell may not be identical to the parent cell transfected with the nucleic acid molecule, e.g., due to mutations or environmental influences that may occur in succeeding generations or integration of the nucleic acid molecule into the host cell genome.

The term “pharmaceutical formulation” or “pharmaceutical composition” refers to a preparation which is in such form as to permit the biological activity of the active ingredient to be effective, and which contains no additional components which are unacceptably toxic to a subject to which the formulation would be administered. The formulation can be sterile.

The terms “administer”, “administering”, “administration”, and the like, as used herein, refer to methods that may be used to enable delivery of active agents (e.g., graft cells, microgels, and/or sirolimus) to the desired site of biological action.

As used herein, the terms “subject” and “patient” are used interchangeably. The subject can be an animal. In some aspects, the subject is a mammal such as a non-human animal (e.g., cow, pig, horse, cat, dog, rat, mouse, monkey or other primate, etc.). In some aspects, the subject is a human.

The term “therapeutically effective amount” refers to an amount effective to treat a disease or disorder in a subject.

Terms such as “treating” or “treatment” or “to treat” or “alleviating” or “to alleviate” refer to therapeutic measures that cure, slow down, lessen symptoms of, and/or halt progression of a diagnosed pathologic condition or disorder. Thus, those in need of treatment include those already diagnosed with or suspected of having the disorder.

As used herein, a “transient” dose or regimen refers to administration that is for a limited time period and is not chronic. For example, transient administration can last no longer than 36 weeks, no longer than 30 weeks, or no longer than 24 weeks.

As used herein a “tapering” dose or regimen refers to administration of an amount that decreases over time. Where the doses in a regimen refer to a range, “tapering” refers to ranges with decreasing upper limits, e.g., a dose regimen that achieves blood trough levels of 9-13 ng/mL for a period, then blood trough levels of 7-11 ng/ml for a period, and then blood trough levels 5-9 ng/ml is a “tapering” regimen.

Alternatively, the pharmacologic and/or physiologic effect may be prophylactic, i.e., the effect completely or partially prevents a disease or symptom thereof. In this respect, the disclosed method comprises administering a “prophylactically effective amount” of a drug (e.g., one or more antibodies or antigen-binding fragments thereof). A “prophylactically effective amount” refers to an amount effective, at dosages and for periods of time necessary, to achieve a desired prophylactic result.

Any compositions or methods provided herein can be combined with one or more of any of the other compositions and methods provided herein.

### Transient Sirolimus Dosing

Provided herein are methods of inducing immune tolerance to graft cells using a transient dose of sirolimus. In some aspects, the sirolimus is administered for 36 weeks or less. In some aspects, the sirolimus is administered for 30 weeks or less. In some aspects, the sirolimus is administered for 24 weeks or less.

According to the methods provided herein, sirolimus can be administered for about 1 to about 36 weeks after graft cells are administered to a patient. In some aspects, sirolimus is administered for about 4 to about 36 weeks after graft cells are administered to a patient. In some aspects, sirolimus is administered for about 8 to about 36 weeks after graft cells are administered to a patient. In some aspects, sirolimus is administered for about 10 to about 36 weeks after graft cells are administered to a patient. In some aspects, sirolimus is administered for about 12 to about 36 weeks after graft cells are administered to a patient.

According to the methods provided herein, sirolimus can be administered for about 1 to about 30 weeks after graft cells are administered to a patient. In some aspects, sirolimus is administered for about 4 to about 30 weeks after graft cells are administered to a patient. In some aspects, sirolimus is administered for about 8 to about 30 weeks after graft cells are administered to a patient. In some aspects, sirolimus is administered for about 10 to about 30 weeks after graft cells are administered to a patient. In some aspects, sirolimus is administered for about 12 to about 30 weeks after graft cells are administered to a patient.

According to the methods provided herein, sirolimus can be administered for about 1 to about 24 weeks after graft cells are administered to a patient. In some aspects, sirolimus is administered for about 4 to about 24 weeks after graft cells are administered to a patient. In some aspects, sirolimus is administered for about 8 to about 24 weeks after graft cells are administered to a patient. In some aspects, sirolimus is administered for about 10 to about 24 weeks after graft cells are administered to a patient. In some aspects, sirolimus is administered for about 12 to about 24 weeks after graft cells are administered to a patient.

In some aspects, the sirolimus is administered at a dose that maintains sirolimus blood trough level of about 4 ng/ml to about 16 ng/ml. The blood trough level of about 4 ng/ml to about 16 ng/ml is maintained for an initial period of about 12 weeks or less, e.g., about 1 week to about 12 weeks, about 2 weeks to about 12, weeks, about 3 weeks to about 12 weeks, about 4 weeks to about 12 weeks, about 5 weeks to about 12 weeks, about 6 weeks to about 12 weeks, about 7 weeks to about 12 weeks, about 8 weeks to about 12 weeks, about 9 weeks to about 12 weeks, about 10 weeks to about 12 weeks, or about 11 weeks to about 12 weeks.

In some aspects, the sirolimus is administered at a dose that maintains sirolimus blood trough level of about 4 ng/ml to about 15 ng/ml. The blood trough level of about 4 ng/mL to about 15 ng/ml is maintained for an initial period of about 12 weeks or less, e.g., about 1 week to about 12 weeks, about 2 weeks to about 12, weeks, about 3 weeks to about 12 weeks, about 4 weeks to about 12 weeks, about 5 weeks to about 12 weeks, about 6 weeks to about 12 weeks, about 7 weeks to about 12 weeks, about 8 weeks to about 12 weeks, about 9 weeks to about 12 weeks, about 10 weeks to about 12 weeks, or about 11 weeks to about 12 weeks.

In some aspects, the sirolimus is administered at a dose that maintains sirolimus blood trough level of about 9 ng/ml to about 16 ng/mL. The blood trough level of about 9 ng/ml to about 16 ng/ml is maintained for an initial period of about 12 weeks or less, e.g., about 1 week to about 12 weeks, about 2 weeks to about 12, weeks, about 3 weeks to about 12 weeks, about 4 weeks to about 12 weeks, about 5 weeks to about 12 weeks, about 6 weeks to about 12 weeks, about 7 weeks to about 12 weeks, about 8 weeks to about 12 weeks, about 9 weeks to about 12 weeks, about 10 weeks to about 12 weeks, or about 11 weeks to about 12 weeks.

In some aspects, the sirolimus is administered at a dose that maintains sirolimus blood trough level of about 9 ng/ml to about 15 ng/ml. The blood trough level of about 9 ng/mL to about 15 ng/ml is maintained for an initial period of about 12 weeks or less, e.g., about 1 week to about 12 weeks, about 2 weeks to about 12, weeks, about 3 weeks to about 12 weeks, about 4 weeks to about 12 weeks, about 5 weeks to about 12 weeks, about 6 weeks to about 12 weeks, about 7 weeks to about 12 weeks, about 8 weeks to about 12 weeks, about 9 weeks to about 12 weeks, about 10 weeks to about 12 weeks, or about 11 weeks to about 12 weeks.

In some aspects, the sirolimus is administered at a dose that maintains sirolimus blood trough level of about 9 ng/ml to about 13 ng/ml. The blood trough level of about 9 ng/ml to about 13 ng/ml is maintained for an initial period of about 12 weeks or less, e.g., about 1 week to about 12 weeks, about 2 weeks to about 12, weeks, about 3 weeks to about 12 weeks, about 4 weeks to about 12 weeks, about 5 weeks to about 12 weeks, about 6 weeks to about 12 weeks, about 7 weeks to about 12 weeks, about 8 weeks to about 12 weeks, about 9 weeks to about 12 weeks, about 10 weeks to about 12 weeks, or about 11 weeks to about 12 weeks.

The sirolimus can be administered in a tapering regimen in which multiple doses are administered and each dose is decreased as compared to the previous dose. The sirolimus can be administered in a tapering regimen in which multiple doses are administered, each dose targets a blood trough range, and the upper limit of each range is decreased as compared to the previous dose. For example, the sirolimus can be administered in an initial dose followed by one or more tapering doses. In some aspects, the method comprises an initial dose and at least one tapering dose (e.g., 1 to 10 tapering doses or 1 to 5 tapering doses). In some aspects, the method comprises an initial dose and at least two tapering doses (e.g., 2 to 10 tapering doses or 2 to 5 tapering doses). In some aspects, the method comprises an initial dose and at least three tapering doses (e.g., 3 to 10 tapering doses or 3 to 5 tapering doses).

The initial dose can maintain sirolimus blood trough level of about 4 ng/ml to about 16 ng/mL. The initial dose can maintain sirolimus blood trough level of about 9 ng/ml to about 16 ng/mL. The initial dose can maintain sirolimus blood trough level of about 4 ng/ml to about 15 ng/mL. The initial dose can maintain sirolimus blood trough level of about 9 ng/ml to about 15 ng/mL. The initial dose can maintain sirolimus blood trough level of about 9 ng/ml to about 13 ng/mL. The blood trough level of is the initial dose can be maintained for an initial period of about 12 weeks or less, e.g., about 1 week to about 12 weeks, about 2 weeks to about 12, weeks, about 3 weeks to about 12 weeks, about 4 weeks to about 12 weeks, about 5 weeks to about 12 weeks, about 6 weeks to about 12 weeks, about 7 weeks to about 12 weeks, about 8 weeks to about 12 weeks, about 9 weeks to about 12 weeks, about 10 weeks to about 12 weeks, or about 11 weeks to about 12 weeks.

Then, e.g., after the initial dose, the sirolimus can be administered in one or more tapering doses for one or more tapering periods. For instance, in some aspects, a first tapering dose maintains sirolimus blood trough levels of about 4 ng/ml to about 11 ng/ml, e.g., about 7 ng/ml to about 11 ng/mL. The first tapering period can be about 2 weeks or less, e.g., about 1 week to about 2 weeks. In some aspects, a second tapering dose maintains sirolimus blood trough levels of about 4 ng/mL to about 9 ng/mL, e.g., about 5 ng/ml to about 9 ng/mL. The second tapering period can be about 2 weeks or less, e.g., about 1 week to about 2 weeks. In some aspects, a third tapering dose maintains sirolimus blood trough levels of about 3 ng/ml to about 7 ng/ml. The third tapering period can be about 2 weeks or less, e.g., about 1 week to about 2 weeks. In some aspects, a fourth tapering dose maintains sirolimus blood trough levels of about 1 ng/ml to about 5 ng/mL. The fourth tapering period can be about 2 weeks or less, e.g., about 1 week to about 2 weeks. In some aspects, a fifth tapering dose maintains sirolimus blood trough levels of about 0 ng/mL to about 3 ng/mL. The fifth tapering period can be about 2 weeks or less, e.g., about 1 week to about 2 weeks.

In some aspects, the tapering regimen comprises maintaining sirolimus blood trough levels of about 4 ng/ml to about 11 ng/ml for 2 weeks or less, maintaining sirolimus blood trough levels of about 4 ng/ml to about 9 ng/ml for 2 weeks or less, maintaining sirolimus blood trough levels of about 3 ng/ml to about 7 ng/ml for 2 weeks or less, maintaining sirolimus blood trough levels of about 1 ng/ml to about 5 ng/ml for 2 weeks or less, and/or maintaining sirolimus blood trough levels of about 0 ng/ml to about 3 ng/ml for 2 weeks or less.

In some aspects, the tapering regimen comprises maintaining sirolimus blood trough levels of about 7 ng/ml to about 11 ng/mL for 2 weeks or less, maintaining sirolimus blood trough levels of about 5 ng/ml to about 9 ng/ml for 2 weeks or less, maintaining sirolimus blood trough levels of about 3 ng/ml to about 7 ng/ml for 2 weeks or less, maintaining sirolimus blood trough levels of about 1 ng/ml to about 5 ng/ml for 2 weeks or less, and/or maintaining sirolimus blood trough levels of about 0 ng/ml to about 3 ng/mL for 2 weeks or less.

In some aspects, the tapering period is about 12 weeks or less. For example, the tapering period can be about 1 week to about 12 weeks, about 2 weeks to about 12 weeks, about 3 weeks to about 12 weeks, about 4 weeks to about 12 weeks, about 5 weeks to about 12 weeks, about 6 weeks to about 12 weeks, about 7 weeks to about 12 weeks, about 8 weeks to about 12 weeks, about 9 weeks to about 12 weeks, about 10 weeks to about 12 weeks, or about 11 weeks to about 12 weeks.

Sirolimus can be administered orally. In some aspects, sirolimus is administered as an oral solution. In some aspects, sirolimus is administered as an oral tablet.

In some aspects, sirolimus is administered once daily.

Administration of sirolimus can begin on the same day as the graft cells and the chimeric FasL protein conjugated to a hydrogel are administered. Alternatively, administration of sirolimus can begin prior to the day on which the graft cells and the chimeric FasL protein conjugated to a hydrogel are administered. For example, administration of sirolimus can begin about 5 days prior to the day on which the graft cells and the chimeric FasL protein conjugated to a hydrogel are administered. Administration of sirolimus can begin about 4 days prior to the day on which the graft cells and the chimeric FasL protein conjugated to a hydrogel are administered. Administration of sirolimus can begin about 3 days prior to the day on which the graft cells and the chimeric FasL protein conjugated to a hydrogel are administered. Administration of sirolimus can begin about 2 days prior to the day on which the graft cells and the chimeric FasL protein conjugated to a hydrogel are administered. Administration of sirolimus can begin about 1 day prior to the day on which the graft cells and the chimeric FasL protein conjugated to a hydrogel are administered

### FASL Hydrogels

FasL hydrogels (e.g., microgels) are known in the art. Exemplary FasL hydrogels (e.g., microgels) are provided in WO 2018/165547, which is herein incorporated by reference in its entirely.

As provided herein, a chimeric FasL protein can comprise a FasL moiety and a heterologous moiety.

In some aspects, a FasL moiety in a chimeric FasL protein comprises a fragment of FasL (e.g., human FasL) that binds to TNFRSF6/FAS. TNFRSF6/FAS is a receptor that binds to FasL and transduces an apoptotic signal into cells. In some aspects, the FasL moiety comprises a fragment of FasL (e.g., human FasL) that binds to TNFRSF6B/DcR3. TNFRSF6B/DcR3 is a soluble decoy receptor for FasL. In some aspects, the FasL moiety comprises an apoptosis-inducing fragment of FasL. In some aspects, the FasL moiety comprises the extracellular domain of FasL.

In some aspects, a FasL moiety does not comprise a transmembrane domain. In some aspects, a FasL moiety does not comprise a cytoplasmic domain. In some aspects, a FasL moiety does not comprise a transmembrane domain or a cytoplasmic domain.

In some aspects, a FasL moiety in a chimeric FasL protein is matrix metalloproteinase (MMP) resistant. In some aspects, the FasL moiety lacks MMP sensitive sites. See Yolcu et al, Immunity 17:795-808 (2002). By way of example, the amino acid sequences of SEQ ID NO: 1 and SEQ ID NO:5 are FasL moieties that lack MMP sensitive sites.

In some aspects, a chimeric FasL protein comprises a human FasL moiety comprising amino acids 131-281 of SEQ ID NO:7 or a sequence that is at least 90%, at least 95%, at least 96%, at least 97%, at least 98%, or at least 99% identical to amino acids 131-281 of SEQ ID NO:7. In some aspects, the human FasL moiety comprising a sequence that is at least 90%, at least 95%, at least 96%, at least 97%, at least 98%, or at least 99% identical to amino acids 131-281 of SEQ ID NO:7 is capable of binding to TNFRSF6/FA. In some aspects, the human FasL moiety comprising a sequence that is at least 90%, at least 95%, at least 96%, at least 97%, at least 98%, or at least 99% identical to amino acids 131-281 of SEQ ID NO:7 is capable of inducing apoptosis in FasL-sensitive cells such as effector T cells (Teffs) and/or A20 lymphoma cells. In some aspects, the human FasL moiety comprising a sequence that is at least 90%, at least 95%, at least 96%, at least 97%, at least 98%, or at least 99% identical to amino acids 131-281 of SEQ ID NO:7 is capable of binding to TNFRSF6/FA and is capable of inducing apoptosis in FasL-sensitive cells such as Teffs and/or A20 lymphoma cells.

In some aspects, a chimeric FasL protein comprises a human FasL moiety comprising the amino acid sequence:

(SEQ ID NO: 1)
IGHPSPPPEKKELRKVAHLTGKSNSRSMPLEWEDTYGIVLLSGVK
YKKGGLVINETGLYFVYSKVYFRGQSCNNLPLSHKVYMRNSKYPQ
DLVMMEGKMMSYCTTGQMWARSSYLGAVENLTSADHLYVNVSELS
LVNFEESQTFFGLYKL.

In some aspects, a chimeric FasL protein comprises a rat FasL moiety comprising the amino acid sequence:

(SEQ ID NO: 5)
IANPSTPSETKKPRSVAHLTGNPRSRSIPLEWEDTYGTALISGVK
YKKGGLVINEAGLYFVYSKVYFRGQSCNSQPLSHKVYMRNFKYPG
DLVLMEEKKLNYCTTGQIWAHSSYLGAVENLTVADHLYVNISQLS
LINFEESKTFFGLYKL.

In some aspects, a heterologous moiety in a chimeric FasL protein comprises a protein that is capable of binding to biotin. In some aspects, a heterologous moiety in a chimeric FasL protein comprises streptavidin or a fragment thereof. As used herein “streptavidin or a fragment thereof” refers to full-length streptavidin or a fragment of full-length streptavidin that is capable of binding to biotin. In some aspects, a heterologous moiety in a chimeric FasL protein comprises avidin or a fragment thereof. As used herein “avidin or a fragment thereof” refers to full-length avidin or a fragment of full-length avidin that is capable of binding to biotin. In some aspects, a heterologous moiety in a chimeric FasL protein comprises streptavidin or a fragment thereof or avidin or a fragment thereof.

Fragments of streptavidin or avidin that can be in a chimeric FasL protein as provided herein include “core streptavidin” (“CSA”), a truncated version of the full-length streptavidin polypeptide which can comprise streptavidin residues 13-138, 14-138, 13-139 or 14-139. See, e.g., Pahler et al., J. Biol. Chem., 262:13933-37 (1987). Other truncated forms of streptavidin and avidin that retain strong binding to biotin also can be used. See, e.g. Sano et al, J Biol Chem. 270 (47): 28204-09 (1995) (describing core streptavidin variants 16-133 and 14-138) (U.S. Pat. No. 6,022,951). Mutants of streptavidin and core forms of streptavidin which retain substantial biotin binding activity or increased biotin binding activity also can be used. See, e.g., Chilcoti et al, Proc Natl Acad Sci, 92 (5): 1754-58 (1995), Reznik et al, Nat Biotechnol, 14 (8): 1007-11 (1996). For example, mutants with reduced immunogenicity, such as mutants created by site-directed mutagenesis to remove potential T cell epitopes or lymphocyte epitopes, can be used. See Meyer et al, Protein Sci., 10:491-503 (2001). Likewise, mutants of avidin and core forms of avidin that retain substantial biotin binding activity or increased biotin binding activity also may be used. See Hiller et al, J Biochem, 278:573-85 (1991); and Livnah et al, Proc Natl Acad Sci USA 90:5076-80 (1993). For convenience, in the discussion herein, the terms “avidin” and “streptavidin” (or “SA”) encompass fragments, mutants, and core forms of these molecules.

Avidin and streptavidin are available from commercial suppliers. Moreover, the nucleic acid sequences encoding streptavidin and avidin and the streptavidin and avidin amino acid sequences are known. See, e.g., GenBank Accession Nos. X65082; X03591; NM_205320; X05343; Z21611; and Z21554.

In some aspects, a chimeric FasL protein comprises a streptavidin moiety comprising the amino acid sequence:

(SEQ ID NO: 2)
ITGTWYNQLGSTFIVTAGADGALTGTYESAVGNAESRYVLTGRYD
SAPATDGSGTALGWTVAWKNNYRNAHSATTWSGQYVGGAEARINT
QWLLTSGATEANAWKSTLVGHDTFTKVKPSAASS

In some aspects, a chimeric FasL protein comprises a streptavidin moiety comprising an amino acid sequence that is at least 90%, at least 95%, at least 96%, at least 97%, at least 98%, or at least 99% identical the amino acid sequence of SEQ ID NO: 2.

In some aspects, a chimeric FasL protein comprises a FasL moiety C-terminal to the heterologous moiety (e.g., the streptavidin or a fragment thereof or avidin or a fragment thereof). The FasL moiety located C-terminal to the heterologous moiety (e.g., the streptavidin or a fragment thereof or avidin or a fragment thereof) can be directly C-terminal to the heterologous moiety or can be separated from the heterologous moiety by a linker (e.g., an amino acid linker). In some aspects, a chimeric FasL protein comprises a FasL moiety N-terminal to the heterologous moiety (e.g., the streptavidin or a fragment thereof or avidin or a fragment thereof). The FasL moiety located N-terminal to the heterologous moiety (e.g., the streptavidin or a fragment thereof or avidin or a fragment thereof) can be directly N-terminal to the heterologous moiety or can be separated from the heterologous moiety by a linker (e.g., an amino acid linker).

In some aspects, the FasL moiety and the heterologous moiety in the chimeric FasL protein are fused via an amino acid linker. In some aspects, the linker is a glycine-serine linker. A glycine-serine linker can comprise, for example, the amino acid sequence GGGGSGGGGSG (SEQ ID NO:3).

In some aspects, a chimeric FasL protein comprising a human FasL moiety comprises the amino acid sequence:

(SEQ ID NO: 4)
ITGTWYNQLGSTFIVTAGADGALTGTYESAVGNAESRYVLTGRYD
SAPATDGSGTALGWTVAWKNNYRNAHSATTWSGQYVGGAEARINT
QWLLTSGATEANAWKSTLVGHDTFTKVKPSAASSGGGGSGGGGSG
EFIGHPSPPPEKKELRKVAHLTGKSNSRSMPLEWEDTYGIVLLSG
VKYKKGGLVINETGLYFVYSKVYFRGQSCNNLPLSHKVYMRNSKY
PQDLVMMEGKMMSYCTTGQMWARSSYLGAVENLTSADHLYVNVSE
LSLVNFEESQTFFGLYKL.

In some aspects, a chimeric FasL protein comprising a rat FasL moiety comprises the amino acid sequence:

(SEQ ID NO: 6)
ITGTWYNQLGSTFIVTAGADGALTGTYESAVGNAESRYVLTGRYD
SAPATDGSGTALGWTVAWKNNYRNAHSATTWSGQYVGGAEARINT
QWLLTSGATEANAWKSTLVGHDTFTKVKPSAASSGGGGSGGGGSG
EFIANPSTPSETKKPRSVAHLTGNPRSRSIPLEWEDTYGTALISG
VKYKKGGLVINEAGLYFVYSKVYFRGQSCNSQPLSHKVYMRNFKY
PGDLVLMEEKKLNYCTTGQIWAHSSYLGAVENLTVADHLYVNISQ
LSLINFEESKTFFGLYKL.

In the amino acid sequence of SEQ ID NO:4, amino acids 1-124 correspond to a streptavidin moiety; amino acids 125-135 correspond to a glycine-serine linker, and amino acids 138-188 correspond to a FasL moiety.

As provided herein, a hydrogel (e.g., a microgel) is engineered to display the chimeric FasL protein. For example, a hydrogel (e.g., a microgel) can be biotinylated and bound to a chimeric FasL protein comprising streptavidin or a fragment thereof or avidin or a fragment thereof. Accordingly, in some aspects a biotinylated hydrogel (e.g., microgel) is bound to a chimeric FasL protein via a non-covalent bond between biotin and streptavidin or a fragment thereof or avidin or a fragment thereof.

In some aspects provided herein, a hydrogel (e.g., a microgel) comprises polyethylene glycol (PEG). In some aspects, the hydrogel (e.g., a microgel) comprises maleimide-terminated 4-arm poly (ethylene) glycol (PEG-4MAL) macromers. Such hydrogels (e.g., microgels) can be synthesized by microfluidic cross-linking. See Headen et al, Advanced Materials, 26:3003-3008 (2014). The PEG-4MAL platform enables stoichiometric, covalent incorporation of thiol-containing molecules, and provides improved cross-linking efficiency for formation of structurally defined hydrogels. See Phelps et al, Advanced Materials, 24:64-70, 62 (2012).

In some aspects, a hydrogel (e.g., microgel) is about 125 microns to about 175 microns in size. In some aspects, a hydrogel (e.g., microgel) is about 150 microns.

Biotinylated hydrogels (e.g., microgels) can be produced by reacting biotin-PEG-thiol with PEG-4MAL macromer, and generating 150 μm diameter microgels cross-linked with dithiothreitol (DTT) via microfluidics cross-linking. The resulting microgels can display covalently-tethered biotin capable of capturing streptavidin (SA) with high affinity.

In some aspects, the FasL in a chimeric FasL protein conjugated to a hydrogel (e.g., microgel) is capable of forming FasL trimers.

In some aspects, a chimeric FasL protein conjugated to a hydrogel (e.g., a microgel) has apoptotic activity.

In some aspects, the hydrogel (e.g., microgel) comprising the chimeric FasL protein encapsulates a graft cell. In some aspects, the hydrogel (e.g., microgel) comprising the chimeric FasL protein does not encapsulate a graft cell.

In some aspects, the hydrogel (e.g., microgel) comprises multiple sites for cross-linking such that additional compounds can be conjugated to the hydrogel (e.g., microgel).

### Graft Cells

As used herein “graft cell” refers to a cell (or tissue or organ comprising a cell), that is administered to a subject in need thereof. In accordance with the methods provided herein, sirolimus and a chimeric FasL protein conjugated to a hydrogel can induce specific immune tolerance to the graft cells. The graft cells can be cells that are derived from a donor, e.g., a deceased donor. The graft cells can also be stem cells or stem cell-derived cells.

Types of graft cells include PBMCs, bone marrow cells, hematopoietic stem cells, stem cells, stem-cell derived cells, mesenchymal stem cells, dendritic cells, dendritic cells incubated with autoantigens, human beta cell products, pancreatic islet cells, alloislets, hepatocytes, and splenocytes.

In some aspects, the graft cells are pancreatic islet cells or insulin producing stem cell-derived pancreatic islet cells. In some aspects, the graft cells are pancreatic islet cells. In some aspects, the graft cells are insulin producing stem cell-derived pancreatic islet cells.

In some aspects, the graft cells are hepatocytes.

Types of graft cells also include islet cells (e.g., pancreatic islet cells), splenocytes, PBMCs, bone marrow cells, mesenchymal stem cells, hematopoietic stem cells, stem cells, induced pluripotent stem cells, human beta cell products, hepatocytes, dendritic cells, macrophages, endothelial cells, cardiac myocytes, and vascular cells, and immune cells, including T cells, etc. In any aspect provided herein, the graft cell can be administered as a preparation of isolated cells or as part of a tissue or organ.

In some aspects provided herein, the graft cell is allogeneic. In some aspects, the grant cell is xenogenic. In some aspects, the graft cell is from a human. In some aspects, the graft cell is from a non-human primate, a dog, a cat, a cow, a sheep, a horse, a rabbit, a mouse, or a rat.

The graft cells for use as provided herein can be graft cells that were obtained from a deceased donor.

In some aspects provided herein, the graft cell is autologous or autogenic (from the subject being treated). For example, an autologous graft cell can be derived from autologous tissue by induced pluripotency and differentiation of the induced pluripotent cells to the desired autologous graft cell. In some aspects, cells from the subject are used to induce immune tolerance to self that has been interrupted in autoimmune disease.

In some aspects, the graft cell is encapsulated by the hydrogel (e.g., microgel) comprising the chimeric FasL protein. In some aspects, the graft cell is not encapsulated by the hydrogel (e.g., microgel) comprising the chimeric FasL protein.

For example, pancreatic islet cells can be administered, along with a tapering and/or transient sirolimus regimen and a chimeric FasL protein conjugated to a hydrogel, to treat diabetes.

In another example, hepatocytes can be administered, along with a sirolimus chimeric FasL protein conjugated to a hydrogel, to treat acute liver failure or liver-based metabolic disorders.

### Uses and Indications

Provided herein are methods of inducing immune tolerance to graft cells. In some aspects, the methods are for preventing or reducing the risks of rejection of cellular or tissue grafts and/or for the treatment of Type I diabetes. The methods can comprise administering the graft cells, a chimeric FasL protein conjugated to a hydrogel, and sirolimus.

In some aspects, the hydrogel (e.g., microgel) comprising the chimeric FasL protein and the graft cell are administered simultaneously. In some aspects, the graft cell is not encapsulated by the hydrogel (e.g., microgel). In such aspects, the graft cell and the hydrogel (e.g., microgel) can be administered in the same pharmaceutical composition or can be administered in separate pharmaceutical compositions.

In some aspects, the graft cell is encapsulated by the hydrogel (e.g., microgel).

In some aspects, the hydrogel and graft cells are administered in a ratio of about 2 hydrogels (e.g., microgels): 1 cell.

In some aspects, the methods of inducing immune tolerance provided herein are for the treatment of Type I diabetes in a human patient. Accordingly, in some aspects, at least 3,000 islet equivalents per kilogram of the patient are administered. In some aspects, at least 5,000 islet equivalents per kilogram of the patient are administered. In some aspects, at least 10,000 islet equivalents per kilogram of the patient are administered. In some aspects, at least 11,000 islet equivalents per kilogram of the patient are administered. In some aspects, about 3,000 to about 20,000 islet equivalents per kilogram of the patient are administered. In some aspects, about 5,000 to about 20,000 islet equivalents per kilogram of the patient are administered. In some aspects, about 10,000 to about 20,000 islet equivalents per kilogram of the patient are administered. In some aspects, about 11,000 to about 20,000 islet equivalents per kilogram of the patient are administered.

In some aspects, the hydrogel and graft cells are administered in a ratio of about 2 hydrogels (e.g., microgels): 1 alloislet.

In some aspects, the methods of inducing immune tolerance provided herein are for the treatment of liver failure, e.g., by administering a therapeutically effective amount of hepatocytes, a chimeric FasL protein conjugated to a hydrogel, and transient sirolimus.

In some aspects of the methods provided herein, the graft cells and the chimeric FasL protein conjugated to a hydrogel are administered to the omentum.

In some aspects, the hydrogels (e.g., microgels), graft cells, and sirolimus are administered or for administration in combination with mesenchymal stem cells (MSCs), an anti-CD20 agent (such as rituximab), or an anti-CD46 agent.

In some aspects, the sirolimus is administered in combination with a prophylactic antiviral (e.g., famciclovir). In some aspects, the sirolimus is administered in combination with an antimicrobial (e.g., sulfamethoxazole and/or trimethoprim). In some aspects, the sirolimus is administered in combination with a prophylactic antiviral (e.g., famciclovir) and an antimicrobial (e.g., sulfamethoxazole and/or trimethoprim).

As used herein administration “in combination” with can refer to simultaneous administration or sequential administration. Simultaneous administration can comprise administration in the same pharmaceutical composition or in separate pharmaceutical compositions.

### EXAMPLES

### Example 1

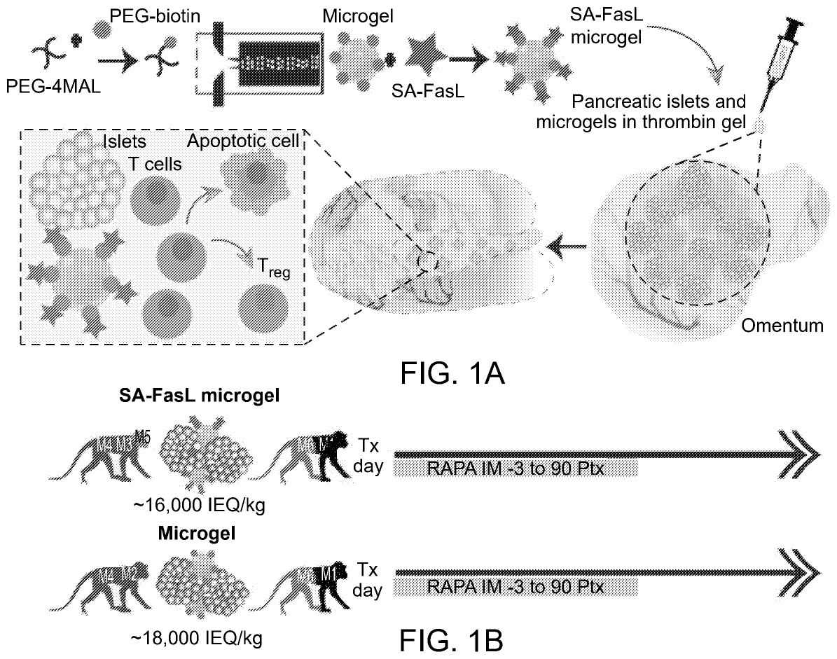

Biotinylated PEG microgels (150 μm diameter) were synthesized by microfluidic cross-linking and functionalized with SA-FasL as previously described (Headon, D M, et al., Nat. Mater. 17:732-739 (2018)). Four diabetic non-human primates (NHP) (FIG. 1B; Tables 1 and 2) underwent transplantation of allogeneic islets (14,800 to 18,700 islet equivalents (IEQ)/kg) mixed with SA-FasL-presenting microgels (150,000 to 200,000 microgels delivering 0.2 mg of SA-FasL) on the omental surface. A 3-month course of sirolimus (rapamycin) monotherapy was used targeting sirolimus blood trough level of 40 ng/ml for the first 2 weeks and then 20 ng/ml until the 3-month time point after transplantation, when sirolimus was discontinued without weaning. Sirolimus levels became undetectable 2 weeks after discontinuation. Three diabetic NHPs (FIG. 1B; Tables 1 and 2) had allogeneic islets co-transplanted with unmodified PEG microgels lacking SA-FasL protein into the omentum under a similar course of sirolimus.

TABLE 1
Summary of NHP Donors and Islet Assessment

| | | Pan- | | Packed | | |
|---|---|---|---|---|---|---|
| | | creas | | Cell | Islet | |
| Cyno | Weight | Weight | | Vol. | Purity | Recipient |
| ID | (kg) | (g) | IEQ | (mL) | (%) | ID |
| M717 | 8.4 | 8.8 | 105,000 | 0.4 | 80 | SA-FasL- |
| | | | | | | Microgel 1 |
| M418 | 7.1 | 8.1 | 93,500 | 0.5 | 50 | SA-FasL- |
| | | | | | | Microgel 2 |
| M118 | 8.5 | 8.7 | 110,200 | 0.3 | 80 | SA-FasL- |
| | | | | | | Microgel 3 |
| M6518 | 6.2 | 6.4 | 80,300 | 0.4 | 65 | SA-FasL- |
| | | | | | | Microgel 4 |
| M1017 | 7.9 | 7.7 | 115,200 | 0.3 | 85 | Microgel 1 |
| M120 | 8.6 | 8.4 | 96,000 | 0.4 | 65 | Microgel 2 |
| M220 | 8.5 | 8.7 | 94,700 | 0.5 | 50 | Microgel 3 |

TABLE 2
Summary of NHP Recipient Characteristics

| | | | Transplant | | |
|---|---|---|---|---|---|
| | | Weight | Dosage | Baseline DSA (MFI) | |
| Animal ID | Cyno ID | (kg) | (IEQ/kg) | Class I | Class II |
| SA-FasL- | M317 | 6.5 | 16,200 | 2326 | 2442 |
| Microgel 1 | | | | | |
| SA-FasL- | M618 | 6.3 | 14,800 | 3628 | 4526 |
| Microgel 2 | | | | | |
| SA-FasL- | M518 | 6.2 | 17,800 | 2027 | 2590 |
| Microgel 3 | | | | | |
| SA-FasL- | M9118 | 4.3 | 18,700 | 2027 | 2590 |
| Microgel 4 | | | | | |
| Microgel 1 | M517 | 5.9 | 19,500 | 727 | 2892 |
| Microgel 2 | M8118 | 4.8 | 20,000 | 162 | 975 |
| Microgel 3 | M8918 | 4.9 | 19,300 | 177 | 1953 |

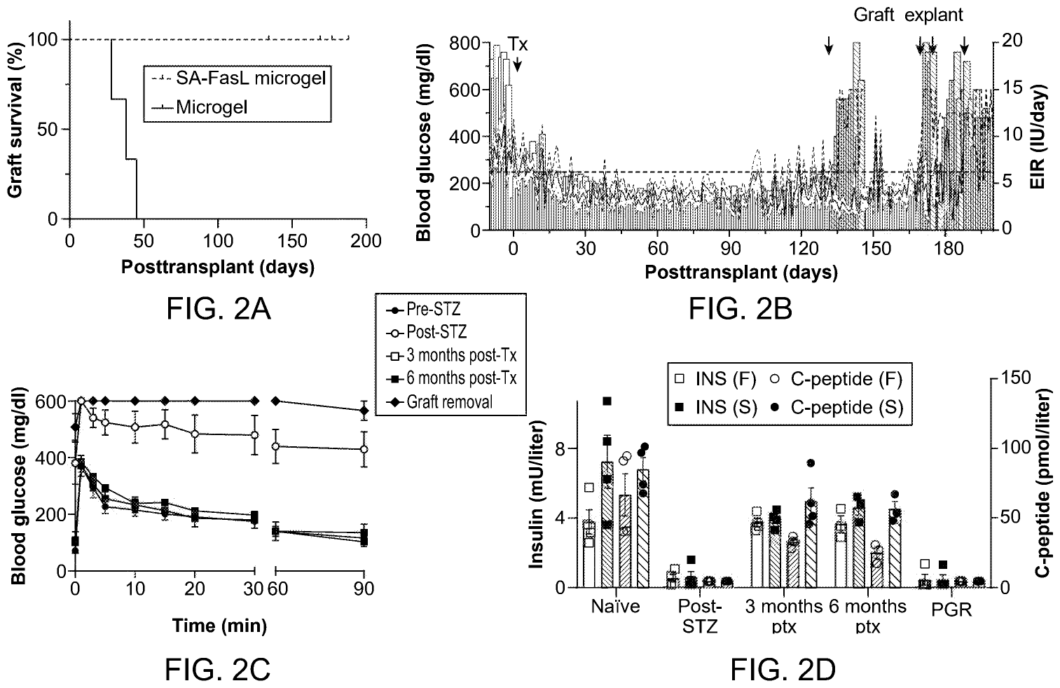

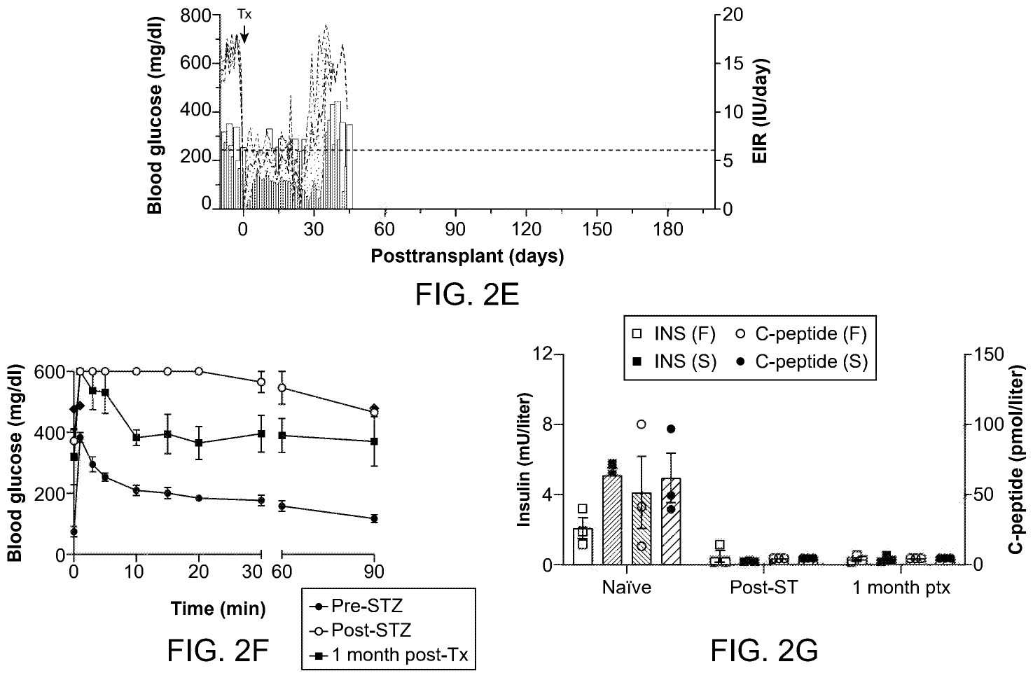

After islet transplantation, prompt glycemic control was achieved and maintained by all NHPs receiving SA-FasL-presenting microgels for observation periods>134, >170, >177, and >188 days (FIGS. 2A and 2B). Animals were terminated with functioning grafts. In contrast, control NHPs, which received an identical sirolimus regiment and had comparable sirolimus plasma levels (P=0.63; FIG. 2D and FIG. 2E) but no SA-FasL, maintained glycemic control for only 21, 27, and 35-days with a mean survival time of 27.7 days (FIG. 2A and FIG. 2E, P=0.010).

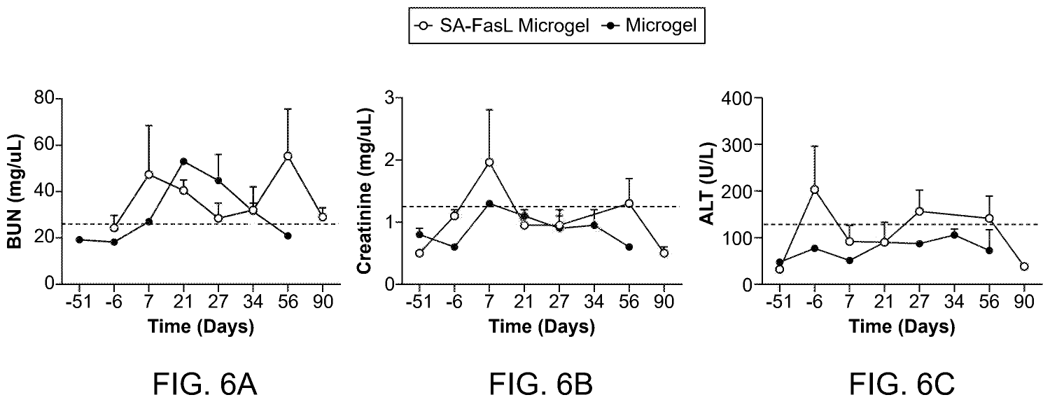

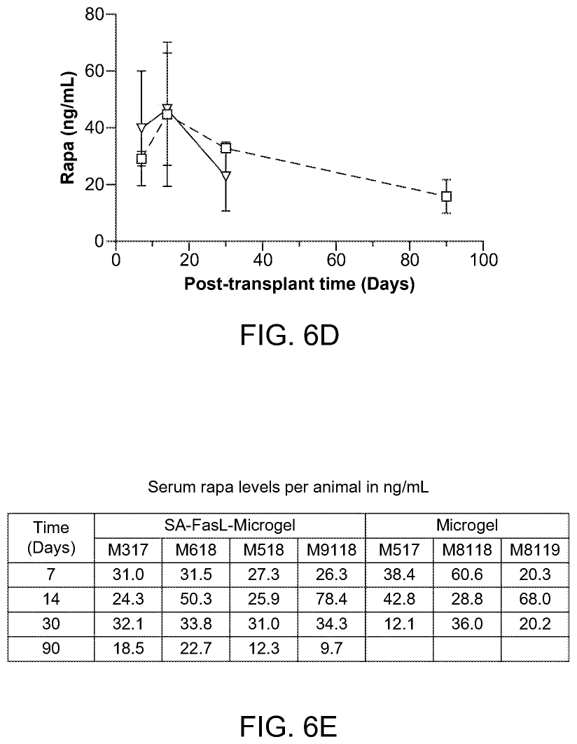

FIG. 2B presents non-fasting blood glucose levels (left axis) and administered external total daily insulin requirement (EIR; right axis) averaged across all SA-FasL-presenting microgel recipients. Before islet transplantation, NHPs in the SA-FasL-microgel group had average non-fasting blood glues levels around 400 mg/dL and required 16 to 22 units of exogenous insulin per day. After transplantation, the fasting blood glucose levels (using intravenous glucose tolerance test (IVGTT) time 0 reading as NHPs were fasted overnight (FIG. 2C) were in the normal range for naïve NHPs (e.g., 37 to 92 mg/dL for ‘M9118), but the random non-fasting glucose levels for some NHPs fluctuated before normal and occasionally >300 mg/dL. Nevertheless, SA-FasL microgel NHPs maintained excellent glycemic control within the normal range for the duration of the study (post-STZ/pre-transplant vs. post-transplant, P=0.0035) (FIG. 2B). Three SA-FasL microgel NHPs required exogenous insulin post-transplantation, but only 10-20% of the pre-transplant dose to maintain postprandial blood glucose levels below 250 mg/dL. Subject 4 (M9118) initially required 1-2 units of insulin per day but developed a fully functioning graft requiring occasional exogenous insulin after 4 months post-transplantation. For SA-FasL microgel NHPs, post-transplant EIR was significantly lower than post-STZ/pre-transplant (P=0.0092). All NHPs experienced initial weight loss post-STZ induction and after transplantation, but they gradually recovered, indicating that post-transplant euglycemia was not due to malnutrition. It is expected that both STZ induction and the transplant surgery will cause immediate weight loss. The continued initial minor weight loss post-transplantation is most likely associated with rapamycin therapy. Results for metabolic markers indicated normal liver and kidney function for SA-FasL microgel NHPs (FIG. 6, blue lines). After surgical removal of islet grafts at the end of the study, SA-FasL microgel NHPs promptly returned to a diabetic state (blood glucose levels: post-transplant vs. post-graft explant, P<0.0001; EIR: post-transplant vs. post-graft, P=0.0042) (FIG. 2B), demonstrating that blood glucose control was due to the graft.

At selected time points, intravenous glucose tolerance tests (IVGTT) were performed to assess glucose disposal kinetics and insulin and C-peptide levels. After glucose bolus challenge, SA-FasL microgel NHPs returned to normal blood glucose levels within 90 minutes with comparable profiles to that of naïve NHPs before diabetes induction [area under the curve (AUC) analysis: pre-STZ [naïve] vs. post-STZ (after STZ induction but prior to transplant), P<0.0001; 3 months post-transplant vs. post-STZ, P<0.0001; 6 months post-transplant vs. post-STZ, P=0.0002) (FIG. 2C). After removal of the graft, blood glucose levels remained elevated and equivalent to pre-transplant diabetic levels (AUC: post-graft removal vs. post-STZ, P=0.0760), demonstrating that the control of blood glucose levels was due to the graft. Consistent with these metrics of graft function, background levels of insulin and C-peptide were measured in the serum of SA-FasL microgel NHPs after STZ administration but before islet transplant and equivalently low levels were evident post-graft removal (insulin: pre-STZ vs. post-STZ, P<0.0001; post-STZ vs. post-graft removal (PGR), P=0.9998; C-peptide: pre-STZ vs. post-STZ, P=0.0060; post-STZ vs. post-graft removal, P=0.4032) (FIG. 2D). SA-FasL microgel NHPs regained insulin and C-peptide expression (insulin: 3 months post-transplant vs. post-STZ, P=0.0022; 6 months post-transplant vs. post-STZ, P=0.0007; C-peptide: 3 months post-transplant vs. post-STZ, P=0.0248; 6 months post-transplant vs. post-STZ, P-0.0230), with levels similar to the pre-diabetic state. Notably, stimulated insulin and C-peptide levels were higher than corresponding fasting levels indicating glucose responsiveness (insulin: P=0.0274, C-peptide: P=0.0149). Glycated hemoglobin (HbA1c) levels for SA-FasL microgel NHPs were well controlled after transplantation, with most (3/4) NPHs with A1C levels below 6% at the end of the study. SA-FasL protein was detected in the serum of all NHPs as early as 1-day after transplantation ranging from 4-56 ng/mL and decreasing to background levels by days 7-14. This result is consistent with prior in vivo measurements of SA-FasL microgels in mice, showing a local half-life of 3.0 days.

FIG. 2E presents random non-fasting blood glucose levels (left axis) and total daily EIR 227 (right axis) averaged across all recipients receiving control microgels (referred to herein as simply “microgel”). Before islet transplantation, microgel NHPs had average non-fasting blood glucose levels around 400 mg/dL while requiring 15-20 units of exogenous insulin per day. After transplantation, microgel NHPs experienced uneventful recovery, and average blood glucose levels returned to the normal range. However, around day 30 post-transplantation, blood glucose levels and required external insulin dose increased and remained elevated at levels comparable to the diabetic pre-transplantation state, indicating graft rejection (blood glucose levels: post-transplant vs. post-rejection, P=0.0050; EIR: post-transplant vs. post-graft rejection, P=0.0441). After transplantation, NHPs initially lost weight but then started gaining weight around day 25. IVGTT showed poor graft function as glucose levels remained elevated following glucose bolus injection (AUC: pre-STZ vs. post-STZ, P=0.0013; 1 month after transplant vs. post-STZ, P=0.1036) (FIG. 2F). Similarly, insulin and C-peptide levels at 1 month post-transplantation remained at pre-transplant diabetic 240 levels (insulin: pre-STZ vs. post-STZ, P<0.0001; 1 month post-transplant vs. post-STZ, 241 P=0.9504; C-peptide: pre-STZ vs. post-STZ, P<0.0134; 1 month post-transplant vs. post-STZ, 242 P>0.9999) (FIG. 2G).

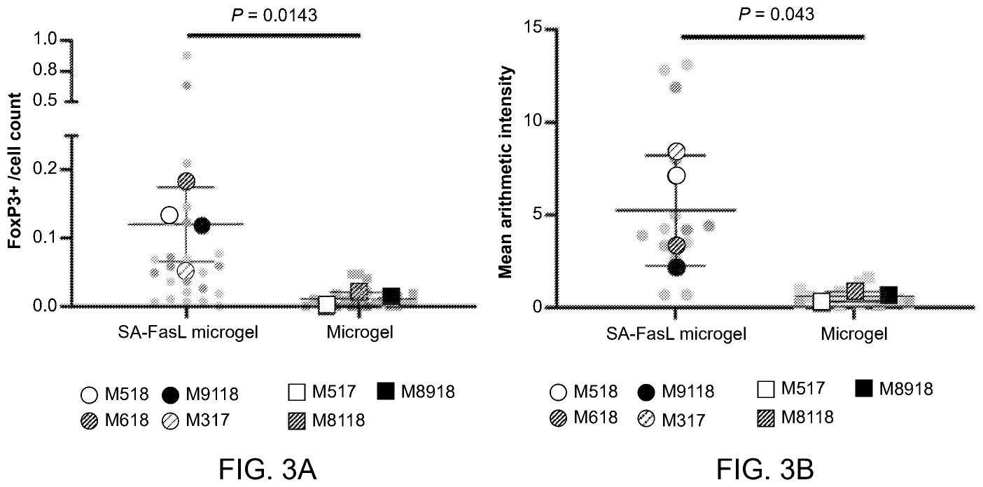

Autopsy pathology showed normal H&E staining for liver, heart, lung, kidney, and intestinal tissue for SA-FasL microgel NHPs. Recipient pancreases contained few small islet-like clusters of endocrine cells but were devoid of insulin-positive β-cells, indicating effective elimination of host B-cells by STZ treatment and confirming that post-transplant glycemic control is attributable to the graft. Histological analyses of the omentum transplant site for SA-FasL microgel NHPs revealed numerous well-granulated, clusters of cells reminiscent of islets with minimal to no infiltrating lymphocytes, suggesting the absence of rejection. Histology of omentum for microgel NHPs revealed islet-like clusters that were infiltrated by lymphocytes, consistent with graft rejection. Immunostaining of the graft site demonstrated well-preserved insulin⁺ structures corresponding to transplanted islets in long-term (day 177) SA-FasL microgel NHPs, whereas sections for microgel control NHPs at rejection (day 21) show structures with loss of insulin staining at the periphery. Importantly, immunostaining analyses of the graft site demonstrated the presence of FoxP3+ (a marker of T_{regs}) cells at a higher frequency (cell count, intensity) in SA-FasL microgel NHPs compared to microgel NHPs (FoxP3⁺ cell counts: P=0.0143; FoxP3⁺ intensity: P=0.043, FIG. 3A-B). This finding is in agreement with studies in diabetic mice demonstrating a pivotal role for T_{regs} in establishing SA-FasL-microgel-induced immune acceptance (Headon, D M, et al., Nat. Mater. 17:732-739 (2018)).

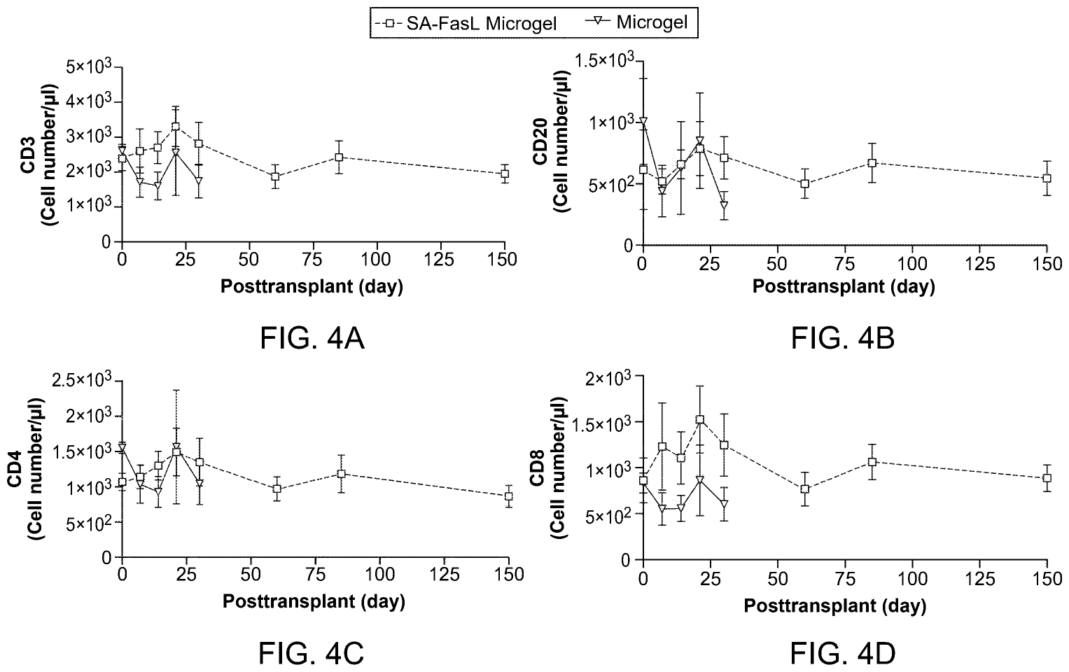

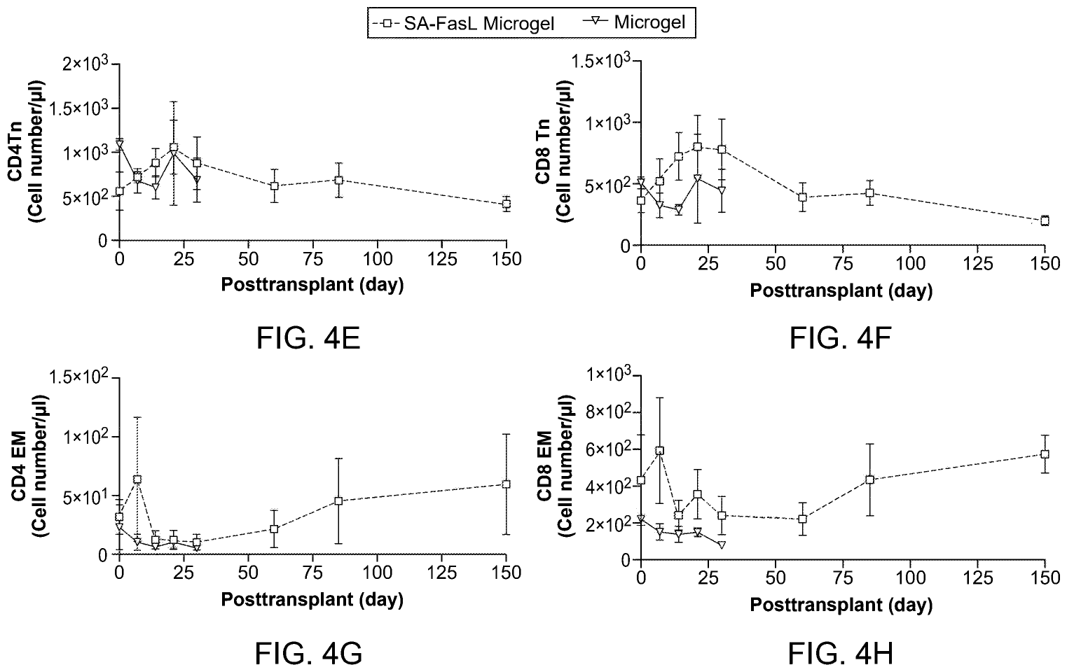

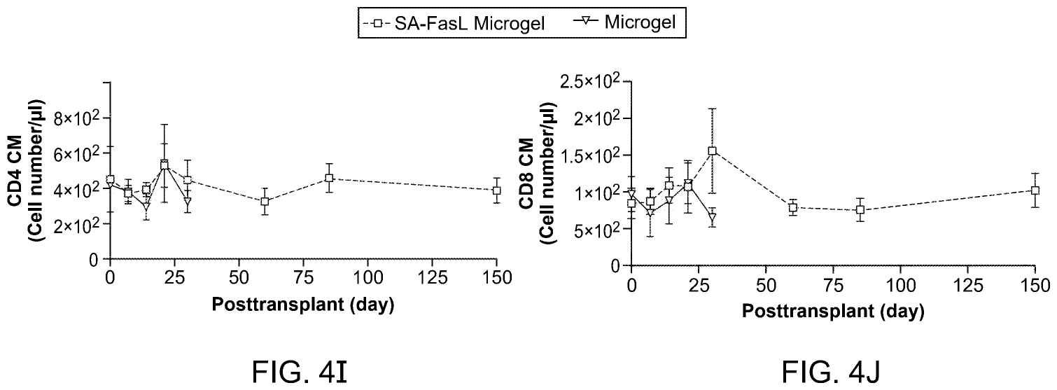

Peripheral blood mononuclear cells (PBMCs) were harvested at selected time points, immunostained, and evaluated by flow cytometry using validated gating strategies. FIG. 4 presents longitudinal profiles for immune cell populations. For both SA-FasL microgel and microgel NHPs, CD20⁺ B cell as well as CD3⁺, CD4⁺ and CD8⁺ T cell levels remained stable over time with minor fluctuations among individual NHPs in both groups (CD20⁺: P=0.5069, P=0.2531; CD3″: P=0.3405, P=0.4959; CD4⁺: P=0.5079, P=0.5142; CD8⁺: P=0.2287, P=0.5277). Similarly, no differences in CD28⁺CD95⁻ CD4⁺ or CD8⁺ naïve, CD28⁺CD95⁺ CD4⁺ or CD8⁺ central memory, CD28⁻CD95⁺CD4⁺ or CD8⁺ effector memory T cells were observed over time (CD4⁺ naïve: P=0.4621, P=0.5102; CD8⁺ naïve: P=0.2524, P-0.5626; CD4⁺ central memory: 283 P=0.5977, P=0.4785; CD8⁺ central memory: P=0.4420, P=0.4790; CD4⁺ effector memory: 284 P=0.4815, P 0.4829; CD8⁺ effector memory: P=0.2442, P=0.0844). The lack of differences in these immune cell populations in systemic circulation over time for SA-FasL microgel NHPs may result from the localization of the immunomodulatory effects of SA-FasL to the graft (Yolcu, E S., at al., J. Immunol. 187:5901-5909 (2011) and Headon, D M, et al., Nat. Mater. 17:732-739 (2018)).

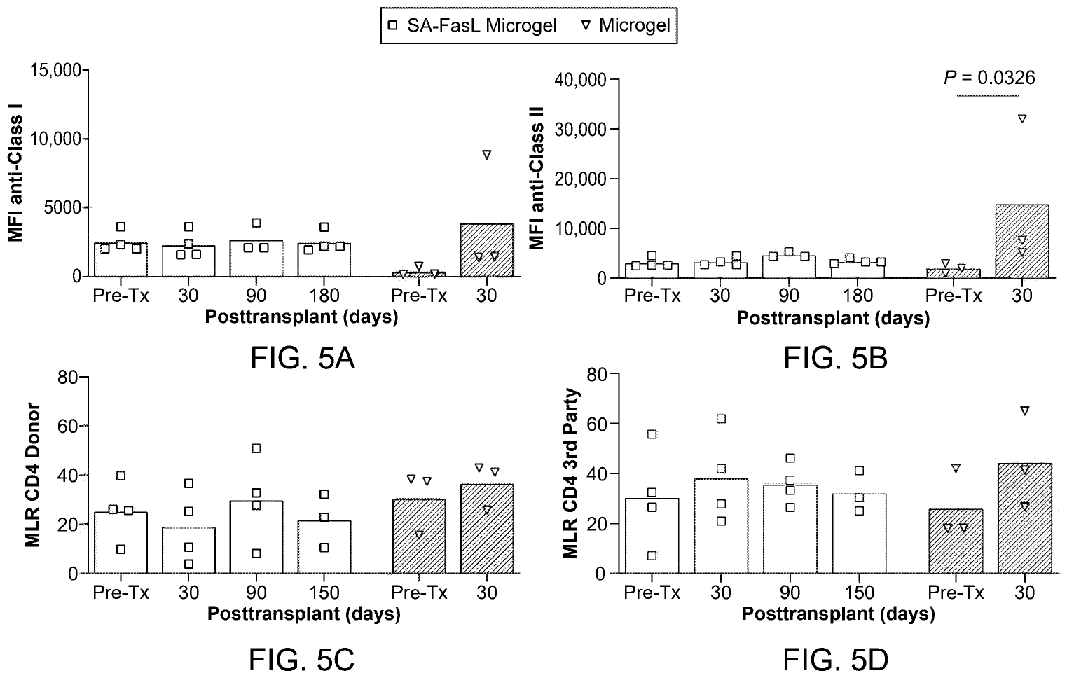

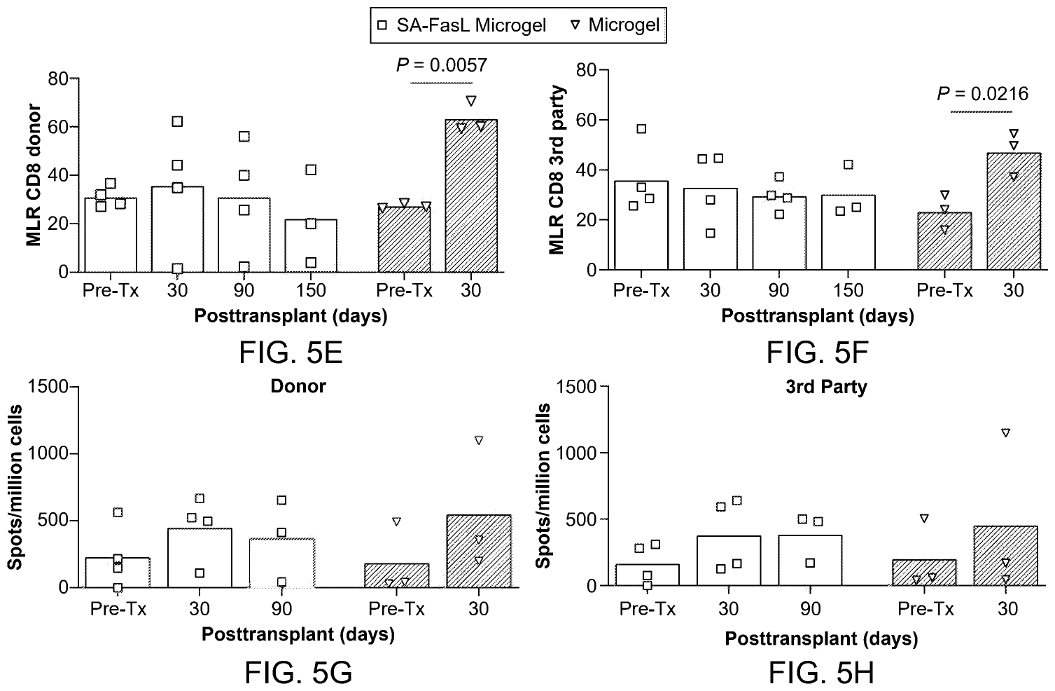

Generation of IgG antibodies against donor MHC antigens was assessed by flow cytometry prior to and at multiple time points after transplantation. No positive donor-specific antibodies to MHC class I (P=0.2904) or MHC class II (P=0.0758) were detected over time in SA-FasL microgel NHPs (FIGS. 5A and 5B). In contrast, there was a significant increase in the titers of antibodies to MHC class II (P=0.0326), but not class I (P=0.3292), in the serum of control microgel NHPs at 1 month after transplantation (FIGS. 5A and 5B). Mixed lymphocyte reactions against donor and third-party CD4″ and CD8⁺ T cells also revealed unchanged responses between pre- and post-transplant conditions for SA-FasL microgel NHPs (CD4⁺ donor: P=0.4200; CD4⁺ third-party: P=0.6949; CD8⁺ donor: P=0.4145; CD8⁺ third-party: P=0.7858) (FIGS. 5C-5F). Although microgel NHPs exhibited unchanged CD4⁺ T cell response against donor (P=0.6082) and third-party (P=0.0555) stimulators, the CD8⁺ T cell compartment showed increased responses towards both donor (P=0.0057) and third-party (P=0.0216) antigens (FIGS. 5C-5F). Enzyme-linked immunospot (ELISpot) assays demonstrated no differences in IFN-γ secretion between pre- and post-transplant time points for SA-FasL microgel (donor: P=0.2485; third-party: P=0.1445) or microgel (donor: P=0.0824; third-party: P=0.1413) NHPs (FIGS. 5G-5H). Multiplexed immunobead assays showed no significant differences between pre- and post-transplantation levels of pro-inflammatory and anti-inflammatory cytokines and chemokines in the serum of SA-FasL microgel and microgel NHPs. Finally, antibodies against SA-FasL protein were not detected in the serum of SA-FasL microgel NHPs at early time points but developed after ˜2 weeks post-transplantation. The majority of antibodies were against the SA domain of the SA-FasL protein. These results show that antibodies against SA-FasL develop ˜2 weeks post-transplantation, when SA-FasL is undetectable systemically, and primarily are against the SA moiety of the chimeric protein. Importantly, the presence of such antibodies did not negatively impact the efficacy of the protocol in sustaining allogeneic islet graft survival.

These results demonstrate that SA-FasL presenting microgels cotransplanted with allogeneic islets are effective in sustaining long-term (>6 months) survival and excellent glycemic control in diabetic NHPs without chronic immunosuppression.

### Example 2

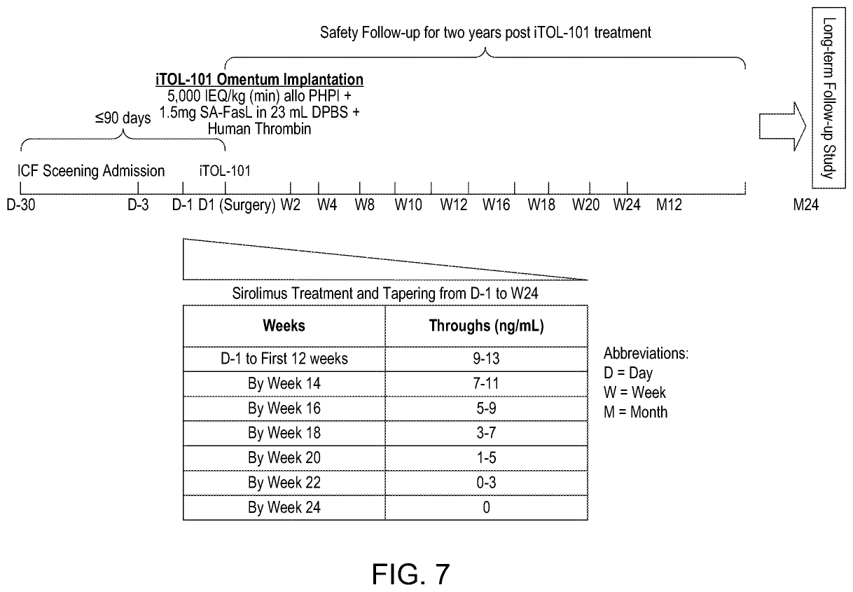

A Phase ½ study is performed to demonstrate the safety, tolerability, and activity of iTOL-101 and short-term administration of sirolimus in adults with poorly controlled diabetes despite intensive diabetes management. A schematic of the study design is depicted in FIG. 7.

iTOL-101

iTOL-101 is made up of two components: 1) the iTOL-100 drug product (SA-FasL microgel) and 2) the allogeneic deceased donor pancreatic islets drug product.

Alloislet dose varies from subject to subject but at least 5,000 islet equivalents (IEQ)/kg of functional islets are delivered for each study subject. Assessment of the total IEQ and minimal dose required for transplant (5,000 IEQ/kg) are determined in accordance with the standard operating procedures (SOPs) used in the National Institutes of Health (NIH) CIT Phase 3 Clinical Trial (ClinicalTrials.gov Identifier: NCT00434811).

An iTOL-100 dose of 0.7 mg SA-FasL in approx. 11 mL of Dulbecco's phosphate-buffered saline is mixed with 350,000 islets (5,000 IEQ/kg) to produce the final delivered dose. The volume of iTOL-100 is adjusted vol: vol with any increases in total amount of pancreatic islets to be implanted.

### Participants

Participants in the study meet the following inclusion criteria:
Adult male and female subjects between 18 and 65 years of age;
Clinical history compatible with Type 1 Diabetes (TID) with onset of disease at <40 years of age and insulin-dependence for >5 years at the time of enrollment;
Absence of stimulated C-peptide (<0.3 ng/mL) in response to a mixed-meal tolerance test (MMTT) measured at 60 and 90 minutes after the start of the meal;
Active intensive diabetes management with ≥2 clinical evaluations during the 12 months prior to study enrollment, under the direction of an endocrinologist or diabetes specialist, and including continuous monitoring of glucose values, administration of ≥3 insulin injections each day, or insulin pump therapy; and
Hypoglycemia unawareness as measured by a Clarke score ≥4 and at least one of the following criteria:
   At least 2 episodes of severe hypoglycemia as defined by American Diabetes Association (ADA) criteria in the 12 months prior to enrollment, despite involvement in intense diabetes management;
   HbA1c>8.0 and glycemic variability >36% (cv);
   HbA1c>7.0 and >8% time below range (blood glucose levels <70 mg/dL)

Participants in the study to not meet any of the following exclusion criteria:
Body mass index (BMI)>30 kg/m² or body weight≤50 kg;
Insulin requirement of >1.0 IU/kg/day or <15 U/day;
HbA1c>10%;
Untreated proliferative diabetic retinopathy;
Blood pressure: systolic >160 mmHg or diastolic >100 mmHg;
Estimated glomerular filtration rate (eGFR)<80 mL/min/1.73 m² calculated using the subject's measured serum creatinine and Chronic Kidney Disease Epidemiology Collaboration (CKD-EPI) equation or Modification of Diet in Renal Disease [MDRD] study estimation formula). Strict vegetarians (vegans) with an eGFR <70 mL/min/1.73 m² are also excluded. The absolute (raw) GFR value is used for subjects with body surface areas >1.73 m²; or
Presence or history of macroalbuminuria (>300 mg/g creatinine).
   iTOL-101 and Sirolimus Administration

The 2 components of iTOL-101 are mixed together at the clinical site and administered via laparoscopic surgery using a 12-French gastrostomy catheter to deliver iTOL-101 to the surface of the greater omentum under pre-transplant sedation and general anesthesia per standard of care. Human recombinant thrombin is added as needed to cover the graft surface with the volume added approximately equal to the volume of the graft implanted to promote adhesion of the islets to the surface of the greater omentum and avoid cell pelleting.

Following iTOL-101 placement on the omentum, an omental pouch (omental flap) is created by folding the omentum over the iTOL-101 implant. The entire procedure, including preparation and delivery of iTOL-101 and formation of omental flap, can be completed in less than 30 minutes.

Sirolimus (mTOR inhibitor) is initiated upon subject admission, 1 to 3 days prior to surgery, for a total of 24 weeks; study subjects are given sirolimus the first 12 weeks adjusted to maintain blood trough levels of 9-13 ng/mL, followed by a 12-week tapering regimen, with a decrease every 2 weeks (+/−7 days) based on blood trough levels. Per sirolimus labeling, prophylactic antiviral (famciclovir), and antimicrobial agents (sulfamethoxazole and trimethoprim) are also be given to subjects for 24 weeks. The sirolimus regimen is as follows:
9-13 ng/mL for the first 12 weeks;
7-11 ng/ml by Week 14;
5-9 ng/mL by Week 16;
3-7 ng/ml by Week 18;
1-5 ng/ml by Week 20;
0-3 ng/ml by Week 22;
0 at Week 24.

### Safety and Efficacy

The following safety endpoints are examined:
Incidence of fever, nausea, vomiting, abdominal pain;
Laparoscopic port site assessments (e.g., bleeding, erythema, pain, tenderness, induration, discharge) post-implantation and at subsequent visits;
Incidence of all adverse events (AEs) reported during the duration of study, which includes any suspected related immunologic reactions;
Incidence of clinically significant grade 3 and 4 laboratory abnormalities;
Evaluation for retinopathy at 3, 6, and 12 months;
Evaluation of eGFR at 3, 6, and 12 months; and
Allosensitization at 12 months.

A review of the safety endpoints is conducted to demonstrate that administration of iTOL-101 and a temporary course of sirolimus is safe.

The following efficacy endpoints are examined.
Number of subjects with insulin independence at 6 months and 12 months;
Number subjects with 50% or greater reduction of insulin requirements compared to baseline at 6 and 12 months;
Number of subjects with peak C-peptide ≥100 pmol/L during an MMTT at 3, 6, and 12 months;
Number of subjects with >70% time in range (blood glucose levels of 70-180 mg/dL) at 3, 6, and 12 months;
Number of subjects with glycemic variability <36% (cv) at 3, 6, and 12 months;
Number of subjects with HbA1c<6.5 or a ≥2% reduction in HbA1c from baseline at 3, 6, and 12 months;
Number of subjects with at least 1 severe hypoglycemic episode at 12 months; and
Number of subjects with reduction >50% or elimination of severe hypoglycemic events at 3, 6, and 12 months.

A review of the efficacy endpoints is conducted to demonstrate that administration of iTOL-101 and a temporary course of sirolimus is effective in treating diabetes.

What is claimed is:

1. A method of inducing immune tolerance to graft cells in a human patient in need thereof, the method comprising administering to the human patient (i) the graft cells, (ii) a chimeric FasL protein conjugated to a hydrogel, and (iii) sirolimus, wherein the sirolimus is administered for 36 weeks or less, optionally for 24 weeks or less.

2. The method of claim 1, wherein sirolimus is administered at an initial dose that maintains sirolimus blood trough level of about 4 ng/mL to about 16 ng/ml or about 9 ng/ml to about 16 ng/mL.

3. The method of claim 1, wherein sirolimus is administered at an initial dose that maintains sirolimus blood trough level of about 4 ng/mL to about 15 ng/ml or about 9 ng/ml to about 15 ng/mL.

4. The method of claim 1, wherein sirolimus is administered at an initial dose that maintains sirolimus blood trough level of about 9 ng/ml to about 13 ng/ml.

5. The method of any one of claims 2-4, wherein the initial dose is administered for an initial period of about 12 weeks or less.

6. The method of any one of claims 1-5, wherein the sirolimus is administered in a tapering regimen for a tapering period, optionally wherein the tapering regimen is administered after the initial dose for the initial period.

7. The method of claim 6, wherein the tapering regimen occurs after the initial dose for the initial period and comprises a first tapering dose administered for a first tapering period and a second tapering dose administered for a second tapering period.

8. The method of claim 7, wherein the first tapering dose maintains sirolimus blood trough levels of about 4 ng/mL to about 11 ng/mL.

9. The method of claim 7, wherein the first tapering dose maintains sirolimus blood trough levels of about 4 ng/ml to about 11 ng/mL.

10. The method of any one of claims 7-9, wherein the first tapering period is (i) about 2 weeks or less or (ii) about 1 week to about 2 weeks.

11. The method of any one of claims 7-10, wherein the second tapering dose maintains sirolimus blood trough levels of about 4 ng/ml to about 9 ng/mL.

12. The method of any one of claims 7-10, wherein the second tapering dose maintains sirolimus blood trough levels of about 5 ng/ml to about 9 ng/mL.

13. The method of any one of claims 7-12, wherein the second tapering period is (i) about 2 weeks or less or (ii) about 1 week to about 2 weeks.

14. The method of any one of claims 7-13, wherein the tapering regimen further comprises a third tapering dose administered for a third tapering period.

15. The method of claim 14, wherein the third tapering dose maintains sirolimus blood trough levels of about 3 ng/ml to about 7 ng/ml.

16. The method of claim 14 or 15, wherein the third tapering period is (i) about 2 weeks or less or (ii) about 1 week to about 2 weeks.

17. The method of any one of claims 14-16, wherein the tapering regimen further comprises a fourth tapering dose administered for a fourth tapering period.

18. The method of claim 17, wherein the fourth tapering dose maintains sirolimus blood trough levels of about 1 ng/ml to about 5 ng/mL.

19. The method of claim 17 or 18, wherein the fourth tapering period is (i) about 2 weeks or less or (ii) about 1 week to about 2 weeks.

20. The method of any one of claims 17-19, wherein the tapering regimen further comprises a fifth tapering dose administered for a fifth tapering period.

21. The method of claim 20, wherein the fifth tapering dose maintains sirolimus blood trough levels of about 0 ng/ml to about 3 ng/mL.

22. The method of claim 20 or 21, wherein the fifth tapering period is (i) about 2 weeks or less or (ii) about 1 week to about 2 weeks.

23. The method of any one of claims 6-22, wherein the tapering regimen comprises maintaining sirolimus blood trough levels of about 4 ng/ml to about 11 ng/mL for 2 weeks or less, maintaining sirolimus blood trough levels of about 4 ng/ml to about 9 ng/mL for 2 weeks or less, maintaining sirolimus blood trough levels of about 3 ng/ml to about 7 ng/mL for 2 weeks or less, maintaining sirolimus blood trough levels of about 1 ng/mL to about 5 ng/mL for 2 weeks or less, and/or maintaining sirolimus blood trough levels of about 0 ng/mL to about 3 ng/mL for 2 weeks or less.

24. The method of any one of claims 6-22, wherein the tapering regimen comprises maintaining sirolimus blood trough levels of about 7 ng/ml to about 11 ng/ml for 2 weeks or less, maintaining sirolimus blood trough levels of about 5 ng/mL to about 9 ng/ml for 2 weeks or less, maintaining sirolimus blood trough levels of about 3 ng/ml to about 7 ng/mL for 2 weeks or less, maintaining sirolimus blood trough levels of about 1 ng/mL to about 5 ng/mL for 2 weeks or less, and/or maintaining sirolimus blood trough levels of about 0 ng/ml to about 3 ng/mL for 2 weeks or less.

25. The method of any one of claims 6-24 wherein the tapering period is (i) about 12 weeks or less or (ii) about 6 weeks to about 12 weeks.

26. The method of any one of claims 1-25, further comprising administering to the subject mesenchymal stem cells, an anti-CD20 agent, and/or an anti-CD47 agent.

27. The method of claim 26, wherein the anti-CD20 agent is rituximab.

28. The method of any one of claims 1-27, wherein the graft cells are selected from PBMCs, bone marrow cells, hematopoietic stem cells, stem cells, stem-cell derived cells, mesenchymal stem cells, dendritic cells, dendritic cells pulsed with autoantigens, human beta cell products, pancreatic islet cells, alloislets, hepatocytes, and splenocytes.

29. The method of claim 28, wherein the graft cells are pancreatic islet cells or insulin producing stem cell-derived pancreatic islet cells.

30. The method of claim 28, wherein the graft cells are hepatocytes.

31. The method of claim 28, wherein the graft cells are stem cells or stem cell-derived cells.

32. The method of any one of claims 1-31, wherein the graft cells are derived from a deceased donor.

33. The method of any one of claims 1-31, wherein the graft cells are allogeneic.

34. The method of any one of claims 1-33, wherein the chimeric FasL protein comprises a FasL moiety and a streptavidin or avidin moiety, optionally wherein the chimeric FasL chimeric protein further comprises a linker between the FasL moiety and the streptavidin or avidin moiety.

35. The method of claim 34, wherein the chimeric FasL protein comprises a FasL moiety and a streptavidin moiety.

36. The method of claim 34 or 35, wherein the FasL moiety is a matrix metalloproteinase resistant FasL protein.

37. The method of any one of claims 1-36, wherein the chimeric FasL protein comprises the amino acid sequence of SEQ ID NO:4.

38. The method of any one of claims 1-37, wherein the chimeric FasL protein is conjugated to the hydrogel via biotin.

39. The method of any one of claims 1-38, wherein the hydrogel is a microgel.

40. The method of claim 39, wherein the microgel is about 125 microns to about 175 microns.

41. The method of claim 40, wherein the microgel is about 150 microns.

42. The method of any one of claims 1-41, wherein the hydrogel is a polyethylene glycol (PEG) microgel.

43. The method of any one of claims 1-42, wherein the hydrogel is engineered to display a biotin moiety.

44. The method of any one of claims 1-43, wherein the graft cells are not encapsulated by the hydrogel.

45. The method of any one of claims 1-44, wherein a 2:1 ratio of hydrogels: graft cells is administered.

46. The method of any one of claims 1-29 and 31-45, wherein the immune tolerance to graft cells is induced to treat type 1 diabetes.

47. The method of claim 46, wherein at least 5,000 islet equivalents per kilogram of the human patient are administered.

48. The method of any one of claims 1-28 and 30-45, wherein the immune tolerance to graft cells is induced to treat liver failure.

49. The method of any one of claims 1-48, wherein the graft cells and the chimeric FasL protein conjugated to a hydrogel are administered to the omentum.

50. The method of any one of claims 1-49, wherein the sirolimus is administered orally.

51. The method of claim 50, wherein the sirolimus is administered as an oral solution.

52. The method of claim 50, wherein the sirolimus is administered as an oral tablet.

53. The method of any one of claims 1-52, wherein the sirolimus is administered once daily.

54. The method of any one of claims 1-53, wherein the sirolimus administration begins on the same day or up to 5 days before the day that the graft cells and the chimeric FasL protein conjugated to a hydrogel are administered to the human subject.

55. The method of any one of claims 1-54, further comprising administering a prophylactic antiviral and an antimicrobial while the sirolimus is administered.

56. The method of claim 55, wherein the prophylactic antiviral is famciclovir.

57. The method of claim 56 or 57, wherein the antimicrobial comprises sulfamethoxazole and/or trimethoprim.

58. Use of a chimeric FasL protein conjugated to a hydrogel for inducing immune tolerance in a human patient according to the method of any one of claims 1-57.

59. A chimeric FasL protein conjugated to a hydrogel for use in inducing immune tolerance according to the method of any one of claims 1-57.


SEQUENCE LISTING (7 sequences)

SEQ ID NO: 1 - protein, 151 aa, synthetic construct
IGHPSPPPEKKELRKVAHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYKL

SEQ ID NO: 2 - protein, 124 aa, synthetic construct
ITGTWYNQLGSTFIVTAGADGALTGTYESAVGNAESRYVLTGRYDSAPATDGSGTALGWTVAWKNNYRNAHSATTWSGQYVGGAEARINTQWLLTSGATEANAWKSTLVGHDTFTKVKPSAASS

SEQ ID NO: 3 - protein, 11 aa, synthetic construct
GGGGSGGGGSG

SEQ ID NO: 4 - protein, 288 aa, synthetic construct
ITGTWYNQLGSTFIVTAGADGALTGTYESAVGNAESRYVLTGRYDSAPATDGSGTALGWTVAWKNNYRNAHSATTWSGQYVGGAEARINTQWLLTSGATEANAWKSTLVGHDTFTKVKPSAASSGGGGSGGGGSGEFIGHPSPPPEKKELRKVAHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYKL

SEQ ID NO: 5 - protein, 151 aa, synthetic construct
IANPSTPSETKKPRSVAHLTGNPRSRSIPLEWEDTYGTALISGVKYKKGGLVINEAGLYFVYSKVYFRGQSCNSQPLSHKVYMRNFKYPGDLVLMEEKKLNYCTTGQIWAHSSYLGAVFNLTVADHLYVNISQLSLINFEESKTFFGLYKL

SEQ ID NO: 6 - protein, 288 aa, synthetic construct
ITGTWYNQLGSTFIVTAGADGALTGTYESAVGNAESRYVLTGRYDSAPATDGSGTALGWTVAWKNNYRNAHSATTWSGQYVGGAEARINTQWLLTSGATEANAWKSTLVGHDTFTKVKPSAASSGGGGSGGGGSGEFIANPSTPSETKKPRSVAHLTGNPRSRSIPLEWEDTYGTALISGVKYKKGGLVINEAGLYFVYSKVYFRGQSCNSQPLSHKVYMRNFKYPGDLVLMEEKKLNYCTTGQIWAHSSYLGAVFNLTVADHLYVNISQLSLINFEESKTFFGLYKL

SEQ ID NO: 7 - protein, 281 aa, Homo sapiens
MQQPFNYPYPQIYWVDSSASSPWAPPGTVLPCPTSVPRRPGQRRPPPPPPPPPLPPPPPPPPLPPLPLPPLKKRGNHSTGLCLLVMFFMVLVALVGLGLGMFQLFHLQKELAELRESTSQMHTASSLEKQIGHPSPPPEKKELRKVAHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYKL